(19) United States
(12) Patent Application Publication
(10) Pub. No.: US 2016/0067212 A1
(43) Pub. Date: Mar. 10, 2016

(54) USE OF INSULIN SIGNALING ANTAGONISTS, OPTIONALLY IN COMBINATION OF TRANSFECTION OF NON-BETA CELLS, FOR INDUCING INSULIN PRODUCTION

(71) Applicant: UNIVERSITE DE GENEVE, Geneva 4 (CH)

(72) Inventors: Pedro HERRERA, Chêne-Bourg (CH); Fabrizio THOREL, Douvaine (FR); Simona CHERA, Bjørndalstrae, Bergen, Hordaland (NO)

(73) Assignee: UNIVERSITE DE GENEVE, Geneva 4 (CH)

(21) Appl. No.: 14/774,166

(22) PCT Filed: Mar. 14, 2014
(86) PCT No.: PCT/IB2014/059779

§ 371 Date: Sep. 10, 2015

Related U.S. Application Data
(60) Provisional application No. 61/787,054, filed on Mar. 15, 2013.

(51) Int. Cl. A61K 31/366; G01N 33/50; C12Q 1/68; G01N 33/68; A61K 38/16; A61K 38/17

(52) U.S. Cl. CPC A61K 31/366; A61K 38/16; A61K 38/1709; C12Q 1/6883; G01N 33/6872; G01N 33/507; A61K 48/00

(57) ABSTRACT

The invention relates to methods of inducing insulin production in non-beta-cells or converting non-beta-cells into insulin producing cells, as well as methods of preventing and/or treating diabetes and methods of predicting the susceptibility of a diabetic subject to a treatment.

### FIELD OF THE INVENTION

The present invention relates to treatment of diabetes, and more particularly to compositions and methods for converting cells other than pancreatic β-cells (“non-β-cells”) into insulin producing cells.

### BACKGROUND OF THE INVENTION

In 2012, it was estimated that diabetes was affecting about 347 million people worldwide and this number is still increasing (World Health Organization's data). Diabetes mellitus occurs throughout the world, but is more prevalent (especially type 2) in the more developed countries. The greatest future increase in prevalence is, however, expected to occur in Asia and Africa, where the majority of sufferers will probably be located by 2030.

Diabetes is a chronic disease that occurs either when the pancreas does not produce enough insulin or when the body cannot effectively use the insulin it does produce. Insulin is a hormone that regulates blood sugar. Hyperglycaemia, or raised blood sugar, is a common effect of uncontrolled diabetes and over time leads to serious damage to many of the body's systems, especially the nerves and blood vessels. Underlying defects lead to a classification of diabetes into two major groups: type 1 and type 2. Type 1 diabetes, or insulin dependent diabetes mellitus (IDDM), arises when patients lack insulin-producing β-cells in their pancreatic glands. Type 2 diabetes, or non-insulin dependent diabetes mellitus (NIDDM), occurs in patients with impaired β-cell function and alterations in insulin action.

The current treatment for type 1 diabetic patients is the injection of insulin, while the majority of type 2 diabetic patients are treated with agents that stimulate β-cell function or with agents that enhance the tissue sensitivity of the patients towards insulin. The drugs presently used to treat type 2 diabetes include alpha-glucosidase inhibitors, insulin sensitizers, insulin secretagogues, metformin and insulin.

An alternative therapeutic approach for treating diabetes would consist of cell replacement-based therapy. However, this method is facing the difficulty of supplying vast numbers of compatible functioning insulin-producing β-cells. One way to increase the number of insulin producing β-cells could be through the reprogramming of alternative endogenous cell types within individual patients. Recent studies reveal significant plasticity between pancreatic at and β-cells under certain induced conditions, implying a potential route to insulin production by transformed α cells. In a near-total β-cell destruction and regeneration model in adult mice, a proportion of new insulin producing cells were produced from α cells via a bi-hormonal glucagon+insulin+transitional state (Thorel et al, 2010, Nature 464: 1149-1154). Despite progress in therapy and patient management through lifestyle, diet and drug treatment, a great need still exists for compositions and methods for the successful treatment and management of diabetes. A better understanding of the potential to exploit plasticity between cells, including pancreatic α cells and β cells, and the agents that may facilitate such plasticity, would result in new therapeutic strategies with enhanced treatment potential and improved quality of life for sufferers.

S961, an insulin receptor antagonist causes hyperglycemia, hyperinsulinemia, insulin-resistance and depletion of energy stores in rats (Vikram and Jena, 2010, Biochem Biophys Res Commun 398: 260-265).

Wortmannin, a steroid metabolite of the fungi Penicillium funiculosum is a specific, covalent inhibitor of phosphoinositide 3-kinases (PI3K). Wortmannin is being clinically tested in relapsing multiple sclerosis in patients treated with interferon β-1a. A derivative of wortmannin, PX-866, has been shown to be a novel, potent, irreversible, inhibitor of PI3K with efficacy when delivered orally. PX-866 is currently in clinical trials including standalone and combination therapy in major human cancers.

### SUMMARY OF THE INVENTION

A first aspect of the invention provides a method of inducing insulin production in non-β-cells comprising the step of stimulating the insulin production of non-β-cells expressing at least one transcription factor characteristic of pancreatic β-cells by blocking the insulin signaling pathway.

A second aspect of the invention relates to a method of converting non-β-cells into insulin producing cells comprising the step of stimulating the insulin production of non-β-cells expressing at least one transcription factor characteristic of pancreatic β-cells by blocking the insulin signaling pathway.

A third aspect of the invention relates to a method of preventing and/or treating diabetes comprising the administration of a therapeutically effective amount of an antagonist of the insulin signaling pathway to a subject in need thereof.

A fourth aspect of the invention relates to a method of preventing and/or treating diabetes in a subject in need thereof comprising auto-grafting or allo-grafting of non-β-cells modified by transfection of a nucleic acid encoding at least one transcription factor characteristic of pancreatic β-cells in combination with an antagonist of the insulin signaling pathway.

A fifth aspect of the invention concerns the use of an antagonist of the insulin signaling pathway in the manufacture of a medicament for the treatment and/or prevention of diabetes.

A sixth aspect of the invention is a use of non-β-cells modified by transfection of a nucleic acid encoding at least one transcription factor characteristic of pancreatic β-cells in combination with an antagonist of the insulin signaling pathway in the manufacture of a medicament for preventing and/or treating diabetes.

A seventh aspect of the invention resides in an antagonist of the insulin signaling pathway for use in preventing and/or treating diabetes.

An eighth aspect of the invention concerns a composition comprising (i) said antagonist and (ii) non-β-cells modified by transfection of a nucleic acid encoding at least one transcription factor characteristic of pancreatic β-cells, for use in preventing and/or treating diabetes.

A ninth aspect of the invention relates to a method of screening a compound for its ability to inhibit the insulin signaling pathway comprising:
a) exposing non-β-cells expressing at least one transcription factor characteristic of β-cells to a test compound;
b) determining the number of said cells which are insulin producing cells in presence and in absence of the test compound;
c) comparing the two values of number of insulin producing cells determined in step b), wherein a number of insulin producing cells that is higher in presence of the test compound compared to the number determined in absence of the test compound is indicative of a test compound able to inhibit the insulin signaling pathway.

A tenth aspect of the invention provides a method of predicting the susceptibility of a diabetic subject to a treatment of diabetes comprising the administration of a therapeutically effective amount of an antagonist of the insulin signaling pathway, comprising a step of detecting the expression of at least one transcription factor characteristic of pancreatic β-cells in non-β-cells from said subject.

An eleventh aspect of the invention relates to a pharmaceutical composition comprising an antagonist of the insulin signaling pathway and, optionally, non-β-cells modified by transfection of a nucleic acid encoding at least one transcription factor characteristic of pancreatic β-cells.

Other features and advantages of the invention will be apparent from the following detailed description.

### DESCRIPTION OF THE FIGURES

FIGS. 1A-1F show that Pdx1 triggers insulin production in adult α-cells after DT-mediated β-cell ablation. (A) Transgenes used. (B) Experimental design. (C) All α-cell containing islets (YFP+) produce insulin. (D) The number of insulin producing cells is increased in pancreas from αPdx1OE mice after DT. (E) The vast majority of insulin-positive cells derive from adult α-cells expressing Pdx1 after DT. (F) The number of reprogrammed α-cells (YFP+Ins+) is increased in αPdx1OE mice after DT.

FIGS. 2A-2C show that ectopic expression of Pdx1 in adult α-cells inhibits glucagon production but fails to induce insulin production in presence of intact β-cell mass. (A) Experimental design. (B) Pancreatic glucagon content is reduced in αPdx1OE mice. (C) Most α-cells are refractory to Pdx1-mediated insulin production in presence of intact β-cell mass. By contrast, Pdx1 efficiently inhibits glucagon production in the vast majority of α-cells (YFP+).

FIGS. 3A-3D show that mice expressing Pdx1 in adult α-cells after DT-mediated β-cell ablation exhibit increased pancreatic insulin content and require less exogenous insulin. (A-B) After DT, αPdx1OE mice have a tendency toward lower hyperglycemia. (C) αPdx1 mice require less insulin to maintain glycemia below 25 mM. (D) Improved pancreatic insulin content in αPdx1OE mice correlates with a lower insulin pellet requirement.

FIGS. 4A-4C show that ectopic expression of Pdx1 in adult α-cells inhibits glucagon production also after DT-mediated β-cell ablation. (A) The number of glucagon-expressing cells is decreased in αPdx1OE mice after DT. (B) most Pdx1-positive α-cells do not produce glucagon after DT. (C) Pdx1 expression in α-cells decreases pancreatic glucagon content rapidly after DT.

FIGS. 5A-5D show that ectopic Pdx1 expression induces insulin production in α-cells after partial β-cell ablation. (A) Experimental design for streptozotocin (STZ)-mediated β-cell ablation. (B) Experimental design for DT-mediated β-cell ablation in hemizygous RIP-DTR females. (C) Most Pdx1-expressing α-cells produce insulin after STZ-mediated subtotal β-cell removal. (D) Partial (50%) β-cell ablation allows insulin production in some α-cells expressing pdx1.

FIG. 6 shows that insulin signaling is down-regulated after DT-induced β-cell ablation. Most components of the insulin signaling are down-regulated after DT. The site of action of the insulin competitor (S961), IGF1-R receptor antagonist (PPP) and PI3 kinase inhibitor (wortmannin) are depicted.

FIGS. 7A-7B show that Pdx1-expressing α-cells produce insulin upon inhibition of insulin signaling. (A) Experimental design. (B) Pdx1-expressing α-cells produce insulin upon S961 and wortmannin (“Wort.”) administration but not after PPP treatment.

FIG. 8 shows that lack of insulin/IGF1 signaling in α-cells predisposes them to insulin expression. (A) Transgenes used. (B) Experimental design.

FIG. 9 shows that human α-cells can reprogram to insulin production. (A) experimental design. (B) Percentage of non-α-cells which are glucagon⁺/Insulin⁺. (C) Percentage of α-cells which are Insulin⁺/Glucagon⁺.

In the figures, “OE” stands for overexpression, “DT” for diphteria toxin, “Ins” for “insulin”, “Gcg” for glucagon.

### DETAILED DESCRIPTION OF THE INVENTION

The terms “α-cells”, “β-cells”, “δ-cells”, “PP cells” and “ε-cells” as used herewith designate five categories of cells found in the pancreas. “α-cells” or “alpha cells” are endocrine cells in the islets of Langerhans of the pancreas, which make up approximately 35% of the human islet cells (Brissova et al, 2005, J. Histochem. Cytochem. 53(9), 1087-1097) and are responsible for synthesizing and secreting the peptide hormone glucagon, which elevates the glucose levels in the blood. “β-cells” or “beta cells” are another type of cell in the pancreas also located in the islets of Langerhans, which make up approximately 54% of the cells in human islets (Brissova et al, 2005, supra). The primary function of β-cells is to manufacture, store and release insulin, a hormone that brings about effects which reduce blood glucose concentration. β-cells can respond quickly to transient increases in blood glucose concentrations by secreting some of their stored insulin while simultaneously producing more. “δ-cells”, “delta cells” and “D cells” are somatostatin-producing cells, which can be found in the stomach, intestine and the islets of Langerhans in the pancreas. “F cells” or “PP cells” designate pancreatic polypeptide producing cells found in the islets of Langerhans of the pancreas. “Epsilon cells” or “ε-cells” are endocrine cells found in the islets of Langerhans which produce the hormone ghrelin.

The term “non-β-cells” refers to any cells which are not pancreatic β-cells. This term includes pancreatic α-cells (“alpha-cells”), pancreatic δ-cells (“delta-cells”), pancreatic PP cells, ε-cells (“epsilon cells”), neuroendocrine cells associated with the digestive tract such as cells from the liver, cells from the intestine, as well as peripheral cells such as cells from the skin. As used herewith “transcription factors characteristic of β-cells” refer to transcription factors directing pancreatic development and β-cell differentiation as well as those regulating gene expression in the mature β-cell. These transcription factors expressed in β-cells include Pdx-1, Nkx 6.1, Nkx 2.2, Pax 4, Pax 6, MafA, Ngn3, NeuroD1 (M E Cerf 2006, Eur J Endocrinol 155: 671-679). Among those transcription factors, Pdx-1 is considered to be the key transcription factor involved in early pancreatic development, β-cell differentiation and maintenance of the mature β-cell. The activity of the NK-family member and homeodomain protein Nkx 2.2 is necessary for the maturation of β-cells, whereas its distant homologue Nkx 6.1 (NK6 homeobox 1) controls their expansion.

As used herewith “Pdx-1” refers to the human or mouse “pancreatic and duodenum homeobox 1”, also called “Ipf-1”, “Idx-1”, “Iuf-1”, “Mody4”, “Stf-1”, “Pdx-1”. In mice, the Pdx-1 protein has 284 amino acids, its sequence is that disclosed under Genebank accession number (NP_{—}032840.1) (SEQ ID NO: 1) and is encoded by a gene of sequence disclosed under Genebank accession number NM_{—}008814. In humans, Pdx-1 protein has 283 amino acids, its amino acid sequence is that disclosed under Genebank accession number NP_{—}000200.1 (SEQ ID NO: 2) and is encoded by a gene of sequence disclosed under Genebank accession number NG_{—}008183. As used herein, the term Pdx-1 also encompasses species variants, homologues, substantially homologous variants (either naturally occurring or synthetic), allelic forms, mutant forms, and equivalents thereof, including conservative substitutions, additions, deletions therein not adversely affecting the structure or function of the protein. Pdx-1 protein is a transcriptional activator of several genes, including insulin, somatostatin, glucokinase, islet amyloid polypeptide, and glucose transporter type 2 (GLUT2).

As used herewith “Nkx 6.1” refers to the human or mouse “Nk6 homeobox 1”, also called “Nkx6A” or “Nkx6-1”. In mice, Nkx 6.1 protein has 365 amino acids and an amino acid sequence as disclosed in Genebank accession number NP_{—}659204.1 (SEQ ID NO: 3) and is encoded by a gene of sequence disclosed under Genebank accession number NM_{—}144955. In humans, Nkx 6.1 protein has 367 amino acids and an amino acid sequence as disclosed in Genebank accession number NP_{—}006159.2 (SEQ ID NO: 4) and is encoded by a gene of sequence disclosed under Genebank accession number NM_{—}006168. As used herein, the term Nkx 6.1 also encompasses species variants, homologues, substantially homologous variants (either naturally occurring or synthetic), allelic forms, mutant forms, and equivalents thereof, including conservative substitutions, additions, deletions therein not adversely affecting the structure or function of the protein. In the pancreas, Nkx 6.1 is required for the development of β cells and is a potent bifunctional transcription regulator that binds to AT-rich sequences within the promoter region of target genes (Iype et al., 2004, Molecular Endocrinology 18(6). 1363-1375).

As used herewith “Nkx 2.2” refers to the human or mouse “Nk2 homeobox 2”, also called “Nkx2B” or “Nkx2-2”. In mice, Nkx 2.2 protein has 273 amino acids and an amino acid sequence as disclosed in Genebank accession number AAI38160.1 (SEQ ID NO: 5). In humans, Nkx 2.2 protein has 273 amino acids and an amino acid sequence as disclosed in Genebank accession number NP_{—}002500.1 (SEQ ID NO: 6) and is encoded by a gene of sequence disclosed under Genebank accession number NM_{—}002509. As used herein, the term Nkx 2.2 also encompasses species variants, homologues, substantially homologous variants (either naturally occurring or synthetic), allelic forms, mutant forms, and equivalents thereof, including conservative substitutions, additions, deletions therein not adversely affecting the structure or function of the protein. Nkx2.2 is required for cell fate decisions in the pancreatic islet and cell patterning in the ventral neural tube. Nkx2.2 acts as a transcriptional repressor to regulate ventral neural patterning through its interaction with the corepressor Groucho-4 (Grg4) (Muhr et al, 2001, Cell 104: 861-873). This interaction is mediated by a motif called the tinman (TN) domain, which shares sequence homology with the core region of the engrailed homology-1 domain in the transcriptional repressor Engrailed and through which Grg/TLE proteins interact (Jimenez et al, 1997, Genes Dev 11: 3072-3082). In the developing pancreas, Nkx2.2 appears to function as either a transcriptional repressor or an activator, depending on the temporal- or cell-specific environment (Anderson et al, 2009, J Biol Chem 284: 31236-31248).

As used herewith “Pax 4” refers to the human or mouse “paired box gene 4”, also called “MODY9”, “KPD”, or “paired domain gene 4. In mice, Pax 4 protein has 349 amino acids and an amino acid sequence as disclosed in Genebank accession number BAA24516 (SEQ ID NO: 7) and is encoded by a gene of sequence disclosed under Genebank accession number NM_{—}011038. In humans, Pax 4 protein has 350 amino acids and an amino acid sequence as disclosed in Genebank accession number 043316 (SEQ ID NO: 8) and is encoded by a gene of sequence disclosed under Genebank accession number NM_{—}006193. As used herein, the term Pax 4 also encompasses species variants, homologues, substantially homologous variants (either naturally occurring or synthetic), allelic forms, mutant forms, and equivalents thereof, including conservative substitutions, additions, deletions therein not adversely affecting the structure or function of the protein. Pax 4 plays a critical role during fetal development and cancer growth. The Pax 4 gene is involved in pancreatic islet development and mouse studies have demonstrated a role for this gene in differentiation of insulin-producing β cells.

As used herewith “Pax 6” refers to the human or mouse “paired box gene 6”, also called “aniridia type II protein”, “AN2” or “oculorhombin”. In mice, Pax 6 protein has 422 amino acids and an amino acid sequence as disclosed in Genebank accession number AAH36957 (SEQ ID NO: 9) and is encoded by a gene of sequence disclosed under Genebank accession number NM_{—}001244198. In humans, Pax 6 protein has 422 amino acids and an amino acid sequence as disclosed in Genebank accession number NP_{—}000271 (SEQ ID NO: 10) and is encoded by a gene of sequence disclosed under Genebank accession number NM_{—}000280. As used herein, the term Pax 6 also encompasses species variants, homologues, substantially homologous variants (either naturally occurring or synthetic), allelic forms, mutant forms, and equivalents thereof, including conservative substitutions, additions, deletions therein not adversely affecting the structure or function of the protein. Pax 6 has important functions in the development of the eye, nose, central nervous system and pancreas. It is required for the differentiation of pancreatic islet α cells, and competes with PAX4 in binding to a common element in glucagon, insulin and somatostatin promoters.

As used herewith “MafA” refers to “Pancreatic β-cell-specific transcriptional activator”, also called “hMafA” or “RIPE3b1”. In mice, MafA protein has 359 amino acids and an amino acid sequence as disclosed in Genebank accession number NP_{—}919331.1 (SEQ ID NO: 11) and is encoded by a gene of sequence disclosed under Genebank accession number AF097357. In humans, MafA protein has 353 amino acids and an amino acid sequence as disclosed in Genebank accession number NP_{—}963883.2 (SEQ ID NO: 12) and is encoded by a gene of sequence disclosed under Genebank accession number AY083269. As used herein, the term MafA also encompasses species variants, homologues, substantially homologous variants (either naturally occurring or synthetic), allelic forms, mutant forms, and equivalents thereof, including conservative substitutions, additions, deletions therein not adversely affecting the structure or function of the protein. MafA binds to DNA and activates gene transcription of Glut2 and pyruvate carboxylase, and other genes such as Glut2, Pdx-1, Nkx 6.1, GLP-1 receptor, prohormone convertase-1/3 as disclosed in Wang et al (2007, Diabetologia 50(2). 348-358).

As used herewith “Ngn3” refers to the human or mouse “neurogenin 3”, also called “Atoh5”, “Math4B”, “bHLHa7”, or “Neurog3”. In mice, Ngn3 protein has 214 amino acids and an amino acid sequence as disclosed in Genebank accession number NP_{—}033849.3 (SEQ ID NO: 13) and is encoded by a gene of sequence disclosed under Genebank accession number NM_{—}009719. In humans, Ngn3 protein has 214 amino acids and an amino acid sequence as disclosed in Genebank accession number NP_{—}066279.2 (SEQ ID NO: 14) and is encoded by a gene of sequence disclosed under Genebank accession number NM_{—}020999. As used herein, the term Ngn3 also encompasses species variants, homologues, substantially homologous variants (either naturally occurring or synthetic), allelic forms, mutant forms, and equivalents thereof, including conservative substitutions, additions, deletions therein not adversely affecting the structure or function of the protein. Ngn-3 is expressed in endocrine progenitor cells and is required for endocrine cell development in the pancreas and intestine (Wang et al., 2006, N Engl J Med, 355(3):270-80). It belongs to a family of basic helix-loop-helix transcription factors involved in the determination of neural precursor cells in the neuroectoderm (Gradwohl et al., 2000, PNAS 97(4)). Ngn3 protein binds to DNA and activates gene transcription of NeuroD, Delta-like 1 (Dll1), HeyL, insulinoma-assiciated-1 (IA1), Nk2.2, Notch, HesS, Isl1, Somatastain receptor 2 (Sstr2) and other genes as disclosed in Serafimidis et al. (2008, Stem cells, 26:3-16).

As used herewith “NeuroD1” refers to the human or mouse “neurogenic differentiation 1”, also called “Beta2”, “Bhf-1”, “bHLHa73”, or “NeuroD”. In mice, NeuroD1 protein has 357 amino acids and an amino acid sequence as disclosed in Genebank accession number AAH94611 (SEQ ID NO: 15) and is encoded by a gene of sequence disclosed under Genebank accession number NM_{—}010894. In humans, NeuroD1 protein has 356 amino acids and an amino acid sequence as disclosed in Genebank accession number NP_{—}002491 (SEQ ID NO: 16) and is encoded by a gene of sequence disclosed under Genebank accession number NM_{—}002500. As used herein, the term NeuroD1 also encompasses species variants, homologues, substantially homologous variants (either naturally occurring or synthetic), allelic forms, mutant forms, and equivalents thereof, including conservative substitutions, additions, deletions therein not adversely affecting the structure or function of the protein. NeuroD1 protein forms heterodimers with other bHLH proteins and activates transcription of genes that contain a specific DNA sequence known as the E-box. It regulates expression of the insulin gene, and mutations in this gene result in type II diabetes mellitus.

The expression “insulin signaling pathway” or “insulin signal transduction pathway” generally designates the chain of reactions starting from the binding of insulin to its receptor (insulin receptor IR) on the cell surface to the biochemical reactions in the cytoplasm leading to regulation of glucose uptake by the cell.

The term “homologous” applied to a gene variant or a polypeptide variant means a gene variant or a polypeptide variant substantially homologous to a gene or a polypeptide of reference, but which has a nucleotide sequence or an amino acid sequence different from that of the gene or polypeptide of reference, respectively, being either from another species or corresponding to natural or synthetic variants as a result of one or more deletions, insertions or substitutions. Substantially homologous means a variant nucleotide sequence that is at least 80%, at least 85%, at least 90%, at least 95%, at least 96%, at least 97%, at least 98% or at least 99% identical to the nucleotide sequence of a gene of reference or an equivalent gene, i.e. exerting the same function, in another species. Substantially homologous means a variant amino acid sequence that is at least 80%, at least 85%, at least 90%, at least 95%, at least 96%, at least 97%, at least 98% or at least 99% identical to the amino acid sequence of a polypeptide of reference or an equivalent polypeptide, i.e. exerting the same function, in another species. The percent identity of two amino acid sequences or two nucleic acid sequences can be determined by visual inspection and/or mathematical calculation, or more easily by comparing sequence information using a computer program such as Clustal package version 1.83. Variants of a gene may comprise a sequence having at least one conservatively substituted amino acid, meaning that a given amino acid residue is replaced by a residue having similar physiochemical characteristics. Generally, substitutions for one or more amino acids present in the native polypeptide should be made conservatively. Examples of conservative substitutions include substitution of amino acids outside of the active domain(s), and substitution of amino acids that do not alter the secondary and/or tertiary structure of the polypeptide of reference. Examples of conservative substitutions include substitution of one aliphatic residue for another, such as Ile, Val, Leu, or Ala for one another, or substitutions of one polar residue for another, such as between Lys and Arg; Glu and Asp; or Gln and Asn. Other such conservative substitutions, for example, substitutions of entire regions having similar hydrophobicity characteristics, are well known (Kyte et al., 1982, J Mol. Biol., 157: 105-131). Naturally occurring variants are also encompassed by the invention. Examples of such variants are proteins that result from alternate mRNA splicing events or from proteolytic cleavage of the native protein, wherein the native biological property is retained. For example, a “conservative amino acid substitution” may involve a substitution of a native amino acid residue with a non-native residue such that there is little or no effect on the polarity or charge of the amino acid residue at that position. Desired amino acid substitutions (whether conservative or non-conservative) can be determined by those skilled in the art at the time such substitutions are desired.

The term “antagonists” of the insulin signaling pathway is defined as a molecule that antagonizes completely or partially the activity of a biological molecule, in the present context the insulin signaling. The antagonists include “peptidomimetics” defined as peptide analogs containing non-peptidic structural elements, which peptides are capable of mimicking or antagonizing the biological action(s) of a natural parent peptide. A peptidomimetic does no longer have classical peptide characteristics such as enzymatically scissile peptide bonds. The antagonists also include antibodies. “Antagonists” of the insulin signaling pathway include known antagonists of the insulin receptor such as S961, S661 (Schtiffer et al, 2008, Biochem Biophys Res Commun 376:380-383; Vikram and Jena, 2010, Biochem Biophys Res Commun 398: 260-265), and a covalent insulin dimer crosslinked between the two B29 lysines (B29-B′29) (Knusden et al, 2012, PLoS ONE 7, 12, e51972), phosphoinositide 3-kinases (PI3K) inhibitors such as wortmannin or a derivative thereof such as PX-866 ((4S,4aR,5R,6aS,9aR,E)-1-((diallylamino)methylene)-11-hydroxy-4-(methoxymethyl)-4a,6a-dimethyl-2,7,10-trioxo-1,2,4,4a,5,6,6a,7,8,9,9a,10-dodecahydroindeno[4,5-h]isochromen-5-yl acetate), or SF1126 ((8 S,14S,17S)-14-(carboxymethyl)-8-(3-guanidinopropyl)-17-(hydroxymethyl)-3,6,9,12,15-pentaoxo-1-(4-(4-oxo-8-phenyl-4H-chromen-2-yl)morpholino-4-ium)-2-oxa-7,10,13,16-tetraazaoctadecan-18-oate), GDC-0941 (4-(2-(1H-indazol-4-yl)-6-((4-(methylsulfonyl)piperazin-1-yl)methyl)thieno[3,2-d]pyrimidin-4-yl)morpholine), XL-147 (N-(3-(benzo[c][1,2,5]thiadiazol-5-ylamino)quinoxalin-2-yl)-4-methylbenzenesulfonamide), XL-765 (2-amino-8-ethyl-4-methyl-6-(1H-pyrazol-3-yl)pyrido[2,3-d]pyrimidin-7(8H)-one), D-87503 (N-[3-(4-Hydroxyphenyl)pyrido[2,3-b]pyrazin-6-yl]-N′-2-propen-1-ylthiourea), D-106669 (N-Ethyl-N′-[3-[(4-methylphenyl)amino]pyrido[2,3-b]pyrazin-6-yl]urea), GSK-615 ( ), CAL-101 (also called Idelalisib, 5-Fluoro-3-phenyl-2-[(1S)-1-(7H-purin-6-ylamino)propyl]-4(3H)-quinazolinone), NVP-BEZ235 (or BEZ-235, 2-Methyl-2-{4-[3-methyl-2-oxo-8-(3-quinolinyl)-2,3-dihydro-1H-imidazo[4,5-c]quinolin-1-yl]phenyl}propane-nitrile), LY294002 (2-(4-morpholinyl)-8-phenyl-4H-1-benzopyran-4-one) (Sauveur-Michel et al, 2009, Biochem. Soc. Trans. 37, 265-272), Buparlisib (also called BKM-120, 5-[2,6-Di(4-morpholinyl)-4-pyrimidinyl]-4-(trifluoromethyl)-2-pyridinamine), GDC-0032 (1H-Pyrazole-1-acetamide, 4-[5,6-dihydro-2-[3-methyl-1-(1-methylethyl)-1H-1,2,4-triazol-5-yl]imidazo-[1,2-d][1,4]benzoxazepin-9-yl]-α,α-dimethyl-), BAY 80-6946 (5-Pyrimidinecarboxamide, 2-amino-N-[2,3-dihydro-7-methoxy-8-[3-(4-morpholinyl)propoxy]imidazo[1,2-c]quinazolin-5-yl]-), IPI-145 ((S)-β-(1-((9H-purin-6-yl)amino)ethyl)-8-chloro-2-phenylisoquinolin-(2H)-one), BYL-719 ((S)—N1-(4-methyl-5-(2-(1,1,1-trifluoro-2-methylpropan-2-yl)pyridin-4-yl)thiazol-2-yl)pyrrolidine-1,2-dicarboxamide), BGT-226 (8-(6-methoxypyridin-3-yl)-3-methyl-1-(4-(piperazin-1-yl)-3-(trifluoromethyl)phenyl)-1H-imidazo[4,5-c]quinolin-2(3H)-one), PF-04691502 (2-Amino-8-[trans-4-(2-hydroxyethoxy)cyclohexyl]-6-(6-methoxy-3-pyridinyl)-4-methyl-pyrido[2,3-d]pyrimidin-7(8H)-one), GDC-0980 ((S)-1-(4-((2-(2-aminopyrimidin-5-yl)-7-methyl-4-morpholinothieno[3,2-d]pyrimidin-6-yl)methyl)piperazin-1-yl)-2-hydroxy-propan-1-one), GSK-2126458 (2,4-difluoro-N-(2-methoxy-5-(4-(pyridazin-4-yl)quinolin-6-yl)pyridin-3-yl)benzenesulfonamide), PF-05212384 (N-[4-[[4-(Dimethylamino)-1-piperidinyl]carbonyl]phenyl]-N′-[4-(4,6-di-4-morpholinyl-1,3,5-triazin-2-yl)phenyl]urea) (Akinleye et al. 2013, Journal of Hematology & Oncology, 6, 88). Also included are antagonists of the intracellular insulin signaling pathway initiated by insulin binding to the insulin receptor, including the critical nodes in the insulin-signalling network as disclosed in Taniguchi et al (Nature Reviews Molecular Cell Biology, 2006, 7, 85-96) or the targets disclosed in Siddle (Journal Molecular Endocrinology, 2011, 47, R1-R10).

The terms “β-cell ablation” designate herewith the loss of β-cells, either total or partial, in the pancreas by apoptosis or necrosis as obtained using, for instance, diphtheria toxin and streptozotocin, respectively. Massive β-cell ablation can be obtained by homozygous transgenic expression of the diphtheria toxin receptor followed by administration of diphtheria toxin as disclosed in Naglich et al (cell, 1992, 69(6): 1051-1061) or Saito et al (Nat Biotechnol, 2001, 19(8): 746-750). Partial β-cells ablation can be obtained by heterozygous transgenic expression of the diphtheria toxin receptor flowed by administration of diphtheria toxin as above, or by using streptozotocin as disclosed in Lenzen, 2008, Diabetologia 2008; 51: 216-26.

As used herewith the term “diabetes” refers to the chronic disease characterized by relative or absolute deficiency of insulin that results in glucose intolerance. This term covers diabetes mellitus, a group of metabolic diseases in which a person has high blood sugar. As used herewith the term “diabetes” includes “diabetes mellitus type 1”, a form of diabetes mellitus that results from autoimmune destruction of insulin-producing β cells of the pancreas, “diabetes mellitus type 2”, a metabolic disorder that is characterized by high blood glucose in the context of insulin resistance and relative insulin deficiency, “gestational diabetes”, a condition in which women without previously diagnosed diabetes exhibit high blood glucose levels during pregnancy, “neonatal diabetes”, a rare form of diabetes that is diagnosed under the age of six months caused by a change in a gene which affects insulin production and “maturity onset diabetes of the young” (MODY), a rare form of hereditary diabetes caused by a mutation in a single gene. As used herein, “treatment” and “treating” and the like generally mean obtaining a desired pharmacological and physiological effect. The effect may be prophylactic in terms of preventing or partially preventing a disease, symptom or condition thereof and/or may be therapeutic in terms of a partial or complete cure of a disease, condition, symptom or adverse effect attributed to the disease. The term “treatment” as used herein covers any treatment of a disease in a mammal, particularly a human, and includes: (a) preventing the disease from occurring in a subject who may be predisposed to the disease but has not yet been diagnosed as having it such as a preventive early asymptomatic intervention; (b) inhibiting the disease, i.e., arresting its development; or relieving the disease, i.e., causing regression of the disease and/or its symptoms or conditions such as improvement or remediation of damage. In particular, the methods, uses, formulations and compositions according to the invention are useful in the treatment of diabetes and/or in the prevention of evolution of diabetes. When applied to diabetes, prevention of a disease or disorder includes the prevention of the appearance or development of diabetes in an individual identified as at risk of developing diabetes, for instance due to past occurrence of diabetes in the circle of the individual's relatives. Also covered by the terms “prevention/treatment” of diabetes is the stabilization of an already diagnosed diabetes in an individual. By “stabilization”, it is meant the prevention or delay of evolution of diabetes leading to complications such as diabetic ketoacidosis, hyperosmolar nonketotic state, hypoglycemia, diabetic coma, respiratory infections, periodontal disease, diabetic cardiomyopathy, diabetic nephropathy, diabetic neuropathy, diabetic foot, diabetic retinopathy, coronary artery disease, diabetic myonecrosis, peripheral vascular disease, stroke, diabetic encephalopathy.

The term “subject” as used herein refers to mammals. For examples, mammals contemplated by the present invention include human, primates, domesticated animals such as cattle, sheep, pigs, horses, laboratory rodents and the like.

The term “effective amount” as used herein refers to an amount of at least one antagonist, composition or pharmaceutical formulation thereof according to the invention, that elicits the biological or medicinal response in a cell, tissue, system, animal or human that is being sought. In one embodiment, the effective amount is a “therapeutically effective amount” for the alleviation of the symptoms of the disease or condition being treated. In another embodiment, the effective amount is a “prophylactically effective amount” for prophylaxis of the symptoms of the disease or condition being prevented. The term also includes herein the amount of active antagonist sufficient to reduce the progression of the disease, notably to delay, reduce or inhibit the complications of diabetes thereby eliciting the response being sought (i.e. an “inhibition effective amount”).

The term “efficacy” of a treatment according to the invention can be measured based on changes in the course of disease in response to a use or a method according to the invention. For example, the efficacy of a treatment of diabetes can be measured by a stable controlled glucose blood level, and/or periodic monitoring of glycated hemoglobin blood level.

The term “pharmaceutical formulation” refers to preparations which are in such a form as to permit biological activity of the active ingredient(s) to be unequivocally effective and which contain no additional component which would be toxic to subjects to which the said formulation would be administered.

### Methods of Inducing Insulin Production in Cells According to the Invention

In a first aspect, the invention provides a method of inducing insulin production in non-β-cells comprising the step of stimulating the insulin production of non-β-cells expressing at least one transcription factor characteristic of pancreatic β-cells by blocking the insulin signaling pathway.

The non-β-cells expressing at least one transcription factor characteristic of pancreatic β-cells according to the invention may for instance, express said transcription factor either naturally in a subject as a result of a diabetic condition, or non-naturally for instance, as a result of induction by genetic engineering or other means by which those cells express at least one transcription factor characteristic of β-cells.

In a particular embodiment, the invention provides a method of inducing insulin production in non-β-cells comprising the steps of:
a) modifying said non-β-cells by inducing the expression of at least one transcription factor characteristic of pancreatic β-cells;
b) stimulating the insulin production of the modified non-β-cells obtained in step a) by blocking the insulin signaling pathway.

In another aspect, the method of the invention relates to a method of converting non-β-cells into insulin producing cells comprising the step of stimulating the insulin production of non-β-cells already expressing at least one transcription factor characteristic of pancreatic β-cells, by blocking the insulin signaling pathway.

As mentioned above, the non-β-cells expressing at least one transcription factor characteristic of pancreatic β-cells according to the invention may express said transcription factor either naturally in a subject as a result of a diabetic condition or non-naturally as a result of induction by genetic engineering or other means by which those cells express at least one transcription factor characteristic of β-cells.

In one embodiment, the method of the invention relates to a method of converting non-β-cells into insulin producing cells comprising the steps of:
a) modifying said non-β-cells by inducing the expression of at least one transcription factor characteristic of pancreatic β-cells; and
b) stimulating the insulin production of the modified non-β-cells obtained in step a) by blocking the insulin signaling pathway.

In a further embodiment of the methods of the invention, at least 10%, in particular at least 20%, more particularly at least 30%, even more particularly at least 40% of the cells obtained in step b) are insulin producing cells.

In another embodiment of the methods of the invention, the amount of insulin produced by the cells obtained in step b) is sufficient to render a significant improvement in the subject's ability to control blood glucose levels. Blood glucose measurement methods are well-known to those skilled in the art.

In a specific aspect, the method of the invention relates to a method of converting pancreatic α-cells into insulin producing cells comprising the steps of:
a) modifying said α-cells by inducing the expression of at least one transcription factor characteristic of pancreatic β-cells; and
b) stimulating the insulin production of the modified cells obtained in step a) by blocking the insulin signaling pathway,
whereby at least 10%, in particular at least 20%, more particularly at least 30%, even more particularly at least 40% of the cells obtained in step b) are insulin producing cells.

The methods of the invention can be applied ex vivo on isolated cells, cell cultures, tissues or sections thereof including those comprising islets of Langerhans, or in vivo in the whole body of an animal, in particular a non-human mammal such as a laboratory rodent, for instance a mouse.

The methods of the invention can apply to various non-β-cell types including pancreatic α-cells, δ-cells, PP cells, ε-cells, neuroendocrine cells associated with the digestive tract such as cells from the liver, cells from the intestine, as well as peripheral cells such as cells from the skin.

Pancreatic tissue and islet cells including α-cells, β-cells, δ-cells and ε-cells can be isolated according to standard methods in the art including fluorescence activated cell sorting (FACS) of human islet cells (Bramswig et al, 2013, J Clin. Inv. 123, p 1275-1284). Neuroendocrine cells associated with the digestive tract such as cells from the liver, cells from the intestine as well as tissues comprising such cells can be isolated according to standard methods that are well-known to those skilled in the art. Peripheral cells such as cells from the skin as well as tissues comprising such cells can be isolated according to standard methods that are well-known to those skilled in the art.

Transcription factors characteristic of pancreatic β-cells according to the invention include Pdx-1, Nkx 6.1, Nkx 2.2, Pax 4, Pax 6, MafA, Ngn3 and NeuroD1.

In a specific embodiment, step a) of the methods of the invention is carried out by inducing expression of at least one transcription factor characteristic of β-cells selected from the group consisting of Pdx-1, Nkx 6.1, Nkx 2.2, Pax 4, Pax 6, MafA, Ngn3, and NeuroD1, in said non-β-cells, in particular in pancreatic α-cells.

In a more specific embodiment, step a) of the methods of the invention is carried out by inducing expression of Pdx-1 in said non-β-cells, in particular in pancreatic α-cells.

In another specific embodiment, step a) of the methods of the invention is carried out by inducing expression of Pdx-1 in said non-β-cells, such as pancreatic α-cells, by transfecting said cells with a nucleic acid comprising the coding sequence of Pdx-1 gene placed under the control of a constitutive or inducible promoter.

In a specific embodiment, step a) of the methods of the invention is carried out by inducing expression of Nkx 6.1 or Nkx 2.2 in said non-β-cells, in particular in pancreatic α-cells. According to the invention, inducing expression of a transcription factor characteristic of β-cells can be carried out by transfecting non-β-cells with a nucleic acid comprising the coding sequence of said transcription factor's gene placed under the control of a constitutive or inducible promoter.

In the method of the invention, the nucleic acid for transfecting said non-β-cells is in the form of a vector (either a viral or non-viral vector) and is delivered into said cells using standard methods in the art including microbubbles, calcium phosphate-DNA co-precipitation, DEAE-dextran-mediated transfection, polybrene-mediated transfection, electroporation, microinjection, liposome fusion, lipofection, protoplast fusion, retroviral infection, and biolistics.

In a specific embodiment, the step of stimulating the insulin production of the methods of the invention is carried out by β-cells ablation (partial or total) in the pancreatic islets of the tissue comprising said pancreatic non-β-cells, either at the tissue level or in vivo, using, for instance, transgenic expression of the diphtheria toxin receptor followed by administration of diphtheria toxin as disclosed in Naglich et al (cell 1992, 69(6): 1051-1061) or Saito et al (Nat Biotechnol, 2001, 19(8): 746-750), or by using streptozotocin as disclosed in Lenzen (Diabetologia, 2008; 51: 216-226).

In another embodiment, the step of inducing the expression of at least one transcription factor characteristic of pancreatic β-cells is also carried out by β-cells ablation (partial or total) as described above.

In another embodiment, the step of blocking the insulin signaling pathway is carried out ex vivo by contacting said non-β-cells with an antagonist of the insulin signaling pathway.

In an alternative embodiment, the step of blocking the insulin signaling pathway is carried out in vivo by administering an antagonist of the insulin signaling pathway to a diabetic subject.

In further embodiment of the invention, the antagonist of the insulin signaling pathway is an insulin receptor antagonist such as S961, S661, a derivative thereof, or a covalent insulin dimer crosslinked between the two B29 lysines (B29-B′29), or a PI3K inhibitor such as Wortmannin or a derivative thereof such as PX-866, or SF1126, GDC-0941, XL-147, XL-765, D-87503, D-106669, GSK-615, CAL-101, NVP-BEZ235, LY294002, Buparlisib (also called BKM-120), GDC-0032, BAY 80-6946, IPI-145, BYL-719, BGT-226, PF-04691502, GDC-0980, GSK-2126458, PF-05212384, or an antagonist of the intracellular insulin signaling pathway initiated by insulin binding to the insulin receptor.

In a still further embodiment, the antagonist of the insulin signaling pathway is the insulin receptor antagonist S961 of sequence SEQ ID NO: 18.

### Methods of Treatment and Uses According to the Invention

Another aspect of the invention relates to a method of preventing and/or treating diabetes comprising the administration of a therapeutically effective amount of an antagonist of the insulin signaling pathway in a subject in need thereof.

In a specific embodiment of the method of the invention, said antagonist is selected from the group consisting of an insulin receptor antagonist such as S961, S661, a derivative thereof, or a covalent insulin dimer crosslinked between the two B29 lysines (B29-B′29), a PI3K inhibitor such as Wortmannin or a derivative thereof such as PX-866, or SF1126, GDC-0941, XL-147, XL-765, D-87503, D-106669, GSK-615, CAL-101, NVP-BEZ235, LY294002, Buparlisib (also called BKM-120), GDC-0032, BAY 80-6946, IPI-145, BYL-719, BGT-226, PF-04691502, GDC-0980, GSK-2126458, PF-05212384, or an antagonist of the intracellular insulin signaling pathway initiated by insulin binding to the insulin receptor. In a specific embodiment, the methods of preventing and/or treating diabetes according to the invention further comprises auto-grafting or allo-grafting of non-β-cells, in particular pancreatic α-cells, modified by transfection of a nucleic acid encoding at least one transcription factor characteristic of pancreatic β-cells, such as Pdx-1, Nkx 6.1, Nkx 2.2, Pax 4, Pax 6, MafA, Ngn3, or NeuroD1.

Auto-grafting consists in grafting modified cells derived from non-13 cells isolated from the subject to be treated, whereas allo-grafting consists in grafting modified cells derived from non-13 cells isolated from a subject different from the subject to be treated but belonging to the same species.

According to the invention, non-β-cells can be administered to the subject prior to, simultaneously or sequentially to the administration of the antagonist of the insulin signaling pathway.

Non-β-cells useful in the method of preventing and/or treating diabetes comprising auto-grafting or allo-grafting of non-β-cells according to the invention can be various pancreatic non-β-cells including α-cells, δ-cells, PP cells, ε-cells, neuroendocrine cells associated with the digestive tract such as cells from the liver, cells from the intestine, as well as peripheral cells such as cells from the skin.

In another aspect, the invention provides a use of an antagonist of the insulin signaling pathway in the manufacture of a medicament for preventing and/or treating diabetes.

In a specific embodiment of the use of the invention, said antagonist is selected from the group consisting of an insulin receptor antagonist such as S961, S661, or a derivative thereof, or a covalent insulin dimer crosslinked between the two B29 lysines (B29-B′29), a PI3K inhibitor such as Wortmannin or a derivative thereof such as PX-866, or SF1126, GDC-0941, XL-147, XL-765, D-87503, D-106669, GSK-615, CAL-101, NVP-BEZ235, LY294002, Buparlisib (also called BKM-120), GDC-0032, BAY 80-6946, IPI-145, BYL-719, BGT-226, PF-04691502, GDC-0980, GSK-2126458, PF-05212384, or an antagonist of the intracellular insulin signaling pathway initiated by insulin binding to the insulin receptor.

In another embodiment, the use of an antagonist according to the invention is combined with the use of non-β-cells, in particular pancreatic α-cells, modified by transfection of a nucleic acid encoding at least one transcription factor characteristic of pancreatic β-cells, such as Pdx-1, Nkx 6.1, Nkx 2.2, Pax 4, Pax 6, MafA, Ngn3, or NeuroD1, for preventing and/or treating diabetes.

In a further embodiment, the methods of preventing and/or treating diabetes according to the invention and the uses according to the invention are applied to a subject identified according to the method described below for predicting the susceptibility of a diabetic subject to a treatment according to the invention.

In another aspect, the invention provides a method of predicting the susceptibility of a diabetic subject to a treatment comprising the administration of a therapeutically effective amount of an antagonist of the insulin signaling pathway, comprising a step of detecting the expression of at least one transcription factor characteristic of pancreatic β-cells, such as Pdx-1, Nkx 6.1, Nkx 2.2, Pax 4, Pax 6, MafA, Ngn3, or NeuroD1, in non-β-cells from said subject.

Any known method in the art may be used for the determination of the expression of said transcription factor including the determination of the level of transcription factor protein in body fluids and the determination of the level of transcription of said transcription factor's gene. Methods considered are e.g. Enzyme-linked immunosorbent assay (ELISA), Radioimmunoassay (RIA), Enzymoimmunoassay (EIA), mass spectrometry, microarray analysis, and RT-PCR.

In one embodiment of the invention, the ELISA consists of a sandwich array wherein conventional microtiter plates are coated with one type of antibody (“first” antibody”) directed against the protein to be analyzed. The plates are then blocked and the sample or standard is loaded. After the incubation, the first antibody is applied followed by a different type of antibody (“second” antibody), directed against the first antibody, conjugated with a suitable label, e.g. an enzyme for chromogenic detection. Finally the plate is developed with a substrate for the label in order to detect and quantify the label, being a measure for the presence and amount of the protein to analyze. If the label is an enzyme for chromogenic detection, the substrate is a colour-generating substrate of the conjugated enzyme. The colour reaction is then detected in a microplate reader and compared to standards.

Suitable pairs of antibodies (“first” and “second” antibody) are any combination of guinea pig, rat, mouse, rabbit, goat, chicken, donkey or horse. Preferred are monoclonal antibodies, but it is also possible to use polyclonal antibodies or antibody fragments. Suitable labels are chromogenic labels, i.e. enzymes which can be used to convert a substrate to a detectable coloured or fluorescent compound, spectroscopic labels, e.g. fluorescent labels or labels presenting a visible colour, affinity labels which may be developed by a further compound specific for the label and allowing easy detection and quantification, or any other label used in standard ELISA.

Other preferred methods of detection of a protein are radioimmunoassay or competitive immunoassay using a single antibody and chemiluminescence detection on automated commercial analytical robots. Microparticle enhanced fluorescence, fluorescence polarized methodologies, or mass spectrometry may also be used. Detection devices, e.g. microarrays, are useful components as readout systems for the analyzed protein.

The methods for determining the level of expression of a transcription factor characteristic of pancreatic β-cells in non-β-cells also include RT-PCR analysis of said transcription factor's gene.

In a specific embodiment, the step of detecting the expression of a transcription factor characteristic of pancreatic β-cells, in particular Pdx-1, in non-β-cells from said subject, comprises:
a) providing a biological sample from said subject;
b) bringing said sample into contact with an antibody directed against said transcription factor, wherein the contacting is under conditions sufficient for binding the transcription factor present in said sample to said antibody through antigen-antibody interactions;
c) detecting the presence of an antigen-antibody complex,
wherein the presence of said complex is indicative of the expression of said transcription factor in non-β-cells from said subject.

In a particular aspect of the above-embodiment, the antibody directed against said transcription factor is fluorescently labeled and the presence of an antigen-antibody complex is detected via fluorescence detection.

In another embodiment, the step of detecting the expression of a transcription factor characteristic of pancreatic β-cells, in particular Pdx-1, in non-β-cells from said subject, comprises:
a) providing a biological sample, in particular a pancreas biopsy sample, from said subject;
b) extracting total RNA from said sample under a);
c) reverse-transcribing the RNA obtained in step b) into cDNA on which quantitative PCR is carried out using appropriate primers for amplifying said transcription factor's gene;
d) detecting the PCR products obtained in step c) specific for said transcription factor's gene;
wherein the presence of said PCR products is indicative of the expression of said transcription factor's gene in non-β-cells, in particular pancreatic α-cells, from said subject.

In a further embodiment, the step of detecting the expression of a transcription factor characteristic of pancreatic β-cells, in particular Pdx-1, is applied to pancreatic non-β-cells such as α-cells, δ-cells, PP cells, ε-cells, neuroendocrine cells associated with the digestive tract such as cells from the liver, cells from the intestine, or peripheral cells such as cells from the skin.

In the above-mentioned embodiment of the invention, a biological sample includes a tissue biopsy sample, a skin scraping, or a mouth swab.

In a still other embodiment, the method of predicting the susceptibility of a diabetic subject to a treatment comprising the administration of a therapeutically effective amount of an antagonist of the insulin signaling pathway further comprises determining, in said biological sample, the proportion of non-β-cells which express said transcription factor, wherein a proportion of non-β-cells, such as pancreatic α-cells, expressing said transcription factor that is equal or higher than 1%, 2%, 3%, 4%, 5%, 10%, 15% or 20% is indicative of the susceptibility of said subject to a treatment comprising the administration of a therapeutically effective amount of an antagonist of the insulin signaling pathway.

### Methods of Screening According to the Invention

In a still other aspect of the invention is provided a method of screening a compound for its ability to inhibit the insulin signaling pathway comprising:
a) exposing non-β-cells, in particular α-cells, expressing at least one transcription factor characteristic of β-cells to a test compound;
b) determining the number of said cells which are insulin producing cells in presence and in absence of the test compound;
c) comparing the two values of number of insulin producing cells determined in step b), wherein a number of insulin producing cells that is higher in presence of the test compound compared to the number determined in absence of the test compound is indicative of a test compound able to inhibit the insulin signaling pathway.

Any known method may be used for the determination of the number of insulin producing cells, including immunofluorescent staining.

### Agents and Compositions According to the Invention

In one aspect, the invention provides an antagonist of the insulin signaling pathway for use in preventing and/or treating diabetes.

In another aspect, the invention provides a composition comprising an antagonist of the insulin signaling pathway and non-β-cells, in particular pancreatic α-cells, modified by transfection of a nucleic acid encoding at least one transcription factor characteristic of pancreatic β-cells, such as Pdx-1, Nkx 6.1, Nkx 2.2, Pax 4, Pax 6, MafA, Ngn3, or NeuroD1, for use in preventing and/or treating diabetes.

In a specific embodiment of the two above aspects, said antagonist is selected from the group consisting of an insulin receptor antagonist such as S961, S661, or a derivative thereof, or a covalent insulin dimer crosslinked between the two B29 lysines (B29-B′29), a phosphoinositide 3-kinases (PI3K) inhibitor such as Wortmannin or a derivative thereof such as PX-866, or SF1126, GDC-0941, XL-147, XL-765, D-87503, D-106669, GSK-615, CAL-101, NVP-BEZ235, LY294002, Buparlisib (also called BKM-120), GDC-0032, BAY 80-6946, IPI-145, BYL-719, BGT-226, PF-04691502, GDC-0980, GSK-2126458, PF-05212384, or an antagonist of the intracellular insulin signaling pathway initiated by insulin binding to the insulin receptor.

S961 is a peptide of amino acid sequence SEQ ID NO: 18 (wherein the two cysteines are connected by a disulfide bond and wherein said peptide has a carboxylic acid group at the C-terminus).

S661 is a peptide of amino acid sequence SEQ ID NO: 17 (wherein the two cysteines are connected by a disulfide bond and wherein said peptide has an amide group at the C-terminus).

The peptide antagonists S661 and S961 can be synthesized by micro-wave-assisted solid-phase peptide synthesis using the Fmoc strategy as described in Schaffer et al (2008, Biochem Biophys Res Commun 376:380-383) and by biosynthesis in E. coli, respectively.

Wortmannin is a steroid metabolite of the fungi Penicillium funiculosum, it is a specific, covalent inhibitor of phosphoinositide 3-kinase (PI3K) of the following formula:

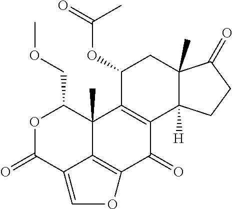 [SMILES: COC[C@H]1OC(=O)c2coc3c2[C@@]1(C)C1=C(C3=O)[C@@H]2CCC(=O)[C@@]2(C)C[C@H]1OC(C)=O]

Derivatives of wortmannin include the analogs described in WO 2011/153495, in particular those of Formula IA or IB:

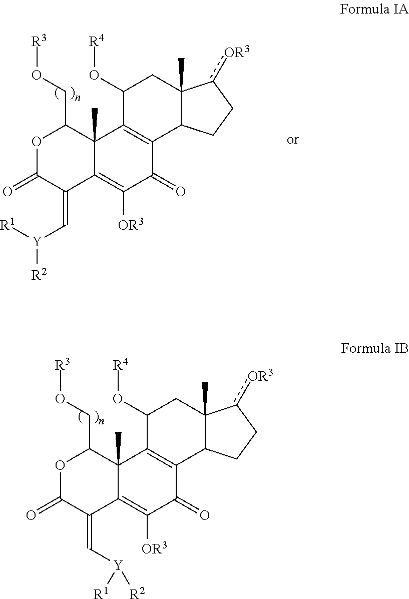

wherein:
--- is an optional bond;
n is 1-6;
Y is a heteroatom;
R¹ and R² are independently selected from an unsaturated alkyl, non-linear alkyl, cyclic alkyl, and substituted alkyl or R¹ and R² together with the atom to which they are attached form a heterocycloalkyl group;
R³ is absent, H, or C1-C6 substituted or unsubstituted alkyl;
R⁴ is (C=0)R⁵, (C=0)OR⁵, (S=0)R⁵, (S02)R⁵, (P03)R⁵, (C=0)NR⁵R⁶;
R⁵ is substituted or unsubstituted C1-C6 alkyl; and
R⁶ is substituted or unsubstituted C1-C6 alkyl.

Derivatives of wortmannin also include the compounds of Formula IIA or IIB:

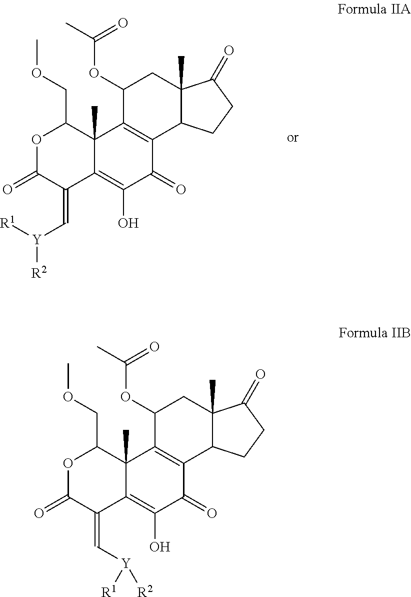

wherein Y is a heteroatom and R¹ and R² are independently selected from an unsaturated alkyl, non-linear alkyl, cyclic alkyl, and substituted alkyl or R¹ and R² together with Y form a heterocycle.

Derivatives of wortmannin also include PX-866 of the following formula:

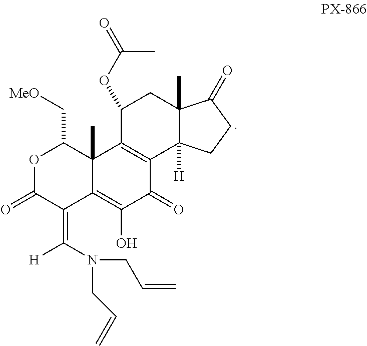 [SMILES: C=CCN(/C=C1/C(=O)O[C@H](COC)[C@@]2(C)C1=C(O)C(=O)C1=C2[C@H](OC(C)=O)C[C@]2(C)C(=O)CC[C@@H]12)CC=C]

According to the invention, said non-β-cells can be used prior to, simultaneously or sequentially to the use of said antagonist of the insulin signaling pathway.

Non-β-cells for use according to the invention can be various pancreatic non-β-cells including α-cells, δ-cells, PP cells, ε-cells, neuroendocrine cells associated with the digestive tract such as cells from the liver, cells from the intestine, as well as peripheral cells such as cells from the skin.

In a further embodiment, the invention provides pharmaceutical compositions and methods for treating a subject, preferably a mammalian subject, and most preferably a human subject who is suffering from diabetes, said pharmaceutical composition comprising the agent according to the invention as described herewith and, optionally, non-β-cells as described herewith.

In particular, said pharmaceutical compositions comprise the agent according to the invention as described herewith and non-β-cells as described herewith.

The agent according to the invention include small molecules (such as antibiotics), peptides, peptidomimetics, chimaeric proteins, natural or unnatural proteins, nucleic acid derived polymers (such as DNA and RNA aptamers, siNAs, siRNAs, shRNAs, PNAs, or LNAs), fusion proteins (such as fusion proteins with insulin receptor antagonizing activities), antibody antagonists (such as neutralizing anti-insulin receptor antibodies).

The invention also provides a pharmaceutical composition comprising an antagonist of the insulin signaling pathway and non-β-cells modified by transfection of a nucleic acid encoding at least one transcription factor characteristic of pancreatic β-cells.

Pharmaceutical compositions or formulations according to the invention may be administered as a pharmaceutical formulation, which can contain an agent according to the invention in any form and non-β-cells as described herewith.

The compositions according to the invention, together with a conventionally employed adjuvant, carrier, diluent or excipient may be placed into the form of pharmaceutical compositions and unit dosages thereof, and in such form may be employed as solids, such as tablets or filled capsules, or liquids such as solutions, suspensions, emulsions, elixirs, or capsules filled with the same, all for oral use, or in the form of sterile injectable solutions for parenteral (including subcutaneous and intradermal) use by injection or continuous infusion. Injectable compositions are typically based upon injectable sterile saline or phosphate-buffered saline or other injectable carriers known in the art. Such pharmaceutical compositions and unit dosage forms thereof may comprise ingredients in conventional proportions, with or without additional active compounds or principles, and such unit dosage forms may contain any suitable effective amount of the active ingredient commensurate with the intended daily dosage range to be employed.

Examples of suitable adjuvants include MPL® (Corixa), aluminum-based minerals including aluminum compounds (generically called Alum), ASO1-4, MF59, CalciumPhosphate, Liposomes, Iscom, polyinosinic:polycytidylic acid (polyIC), including its stabilized form poly-ICLC (Hiltonol), CpG oligodeoxynucleotides, Granulocyte-macrophage colony-stimulating factor (GM-CSF), lipopolysaccharide (LPS), Montanide, PLG, Flagellin, QS21, RC529, IC31, Imiquimod, Resiquimod, ISS, and Fibroblast-stimulating lipopeptide (FSL1). Compositions of the invention may be liquid formulations including, but not limited to, aqueous or oily suspensions, solutions, emulsions, syrups, and elixirs. The compositions may also be formulated as a dry product for reconstitution with water or other suitable vehicle before use. Such liquid preparations may contain additives including, but not limited to, suspending agents, emulsifying agents, non-aqueous vehicles and preservatives. Suspending agents include, but are not limited to, sorbitol syrup, methyl cellulose, glucose/sugar syrup, gelatin, hydroxyethyl cellulose, carboxymethyl cellulose, aluminum stearate gel, and hydrogenated edible fats. Emulsifying agents include, but are not limited to, lecithin, sorbitan monooleate, and acacia. Preservatives include, but are not limited to, methyl or propyl p-hydroxybenzoate and sorbic acid. Dispersing or wetting agents include but are not limited to poly(ethylene glycol), glycerol, bovine serum albumin, Tween®, Span®.

Compositions of the invention may also be formulated as a depot preparation, which may be administered by implantation or by intramuscular injection.

Solid compositions of this invention may be in the form of tablets or lozenges formulated in a conventional manner. For example, tablets and capsules for oral administration may contain conventional excipients including, but not limited to, binding agents, fillers, lubricants, disintegrants and wetting agents. Binding agents include, but are not limited to, syrup, acacia, gelatin, sorbitol, tragacanth, mucilage of starch and polyvinylpyrrolidone. Fillers include, but are not limited to, lactose, sugar, microcrystalline cellulose, maize starch, calcium phosphate, and sorbitol. Lubricants include, but are not limited to, magnesium stearate, stearic acid, talc, polyethylene glycol, and silica. Disintegrants include, but are not limited to, potato starch and sodium starch glycollate. Wetting agents include, but are not limited to, sodium lauryl sulfate. Tablets may be coated according to methods well known in the art.

The compounds of this invention can also be administered in sustained release forms or from sustained release drug delivery systems.

According to a particular embodiment, compositions according to the invention are injectable for subcutaneous, intramuscular or intraperitoneal use or ingestable for oral use.

In another particular aspect, the compositions according to the invention are adapted for delivery by repeated administration.

Further materials as well as formulation processing techniques and the like are set out in Part 5 of Remington's “The Science and Practice of Pharmacy”, 22^{nd} Edition, 2012, University of the Sciences in Philadelphia, Lippincott Williams & Wilkins, which is incorporated herein by reference.

The dosage administered, as single or multiple doses, to an individual will vary depending upon a variety of factors, including pharmacokinetic properties, subject conditions and characteristics (sex, age, body weight, health, size), extent of symptoms, concurrent treatments, frequency of treatment and the effect desired.

### Mode of Administration

Compositions of this invention may be administered in any manner including intravenous injection, intra-arterial, intraperitoneal injection, subcutaneous injection, intramuscular, intrathecal, oral route, cutaneous application, direct tissue perfusion during surgery or combinations thereof.

The compositions of this invention may also be administered in the form of an implant, which allows slow release of the compositions as well as a slow controlled i.v. infusion.

Delivery methods for the composition of this invention include known delivery methods for anti-diabetes drugs such as oral, intramuscular and subcutaneous.

### Combination

According to the invention, the agents and compositions according to the invention, and pharmaceutical formulations thereof can be administered alone or in combination with a co-agent useful in the treatment of diabetes such as insulin, biguanide, sulphonylureas, alpha glucosidase inhibitor, prandial glucose regulators, thiazolidinediones (glitazones), incretin mimetics, DPP-4 inhibitors (gliptins) or in combination with non-β-cells modified by transfection of a nucleic acid encoding at least one transcription factor characteristic of pancreatic β-cells as described herewith.

The invention encompasses the administration of an agent or composition according to the invention and pharmaceutical formulations thereof, wherein said agent or composition is administered to an individual prior to, simultaneously or sequentially with other therapeutic regimens, co-agents useful in the treatment of diabetes, or non-β-cells modified by transfection of a nucleic acid encoding at least one transcription factor characteristic of pancreatic β-cells, in a therapeutically effective amount.

An agent or composition according to the invention, or the pharmaceutical formulation thereof, that is administered simultaneously with said co-agents or said non-β-cells can be administered in the same or different composition(s) and by the same or different route(s) of administration.

According to one embodiment, is provided a pharmaceutical formulation comprising an agent or composition according to the invention, combined with at least one co-agent useful in the treatment of diabetes, and at least one pharmaceutically acceptable carrier.

### Subjects

In an embodiment, subjects according to the invention are subjects suffering from diabetes. In a particular embodiment, subjects according to the invention are subjects suffering from diabetes mellitus type 1, diabetes mellitus type 2, gestational diabetes, neonatal diabetes, or maturity onset diabetes of the young” (MODY).

In a particular embodiment, subjects according to the invention are subjects whose pancreatic cells comprise at least 1%, at least 2%, at least 3%, at least 4%, at least 5%, at least 10%, at least 15% or at least 20% of cells derived from α-cells which express a transcription factor characteristic of β-cells, in particular Pdx-1.

In another particular embodiment, subjects according to the invention are subjects whose pancreatic β-cells decreased by more than 60% compared to non-diabetic subjects. References cited herein are hereby incorporated by reference in their entirety. The present invention is not to be limited in scope by the specific embodiments described herein, which are intended as single illustrations of individual aspects of the invention, and functionally equivalent methods and components are within the scope of the invention. Indeed, various modifications of the invention, in addition to those shown and described herein will become apparent to those skilled in the art from the foregoing description and accompanying drawings. Such modifications are intended to fall within the scope of the appended claims. The invention having been described, the following examples are presented by way of illustration, and not limitation.

### EXAMPLES

The following abbreviations refer respectively to the definitions below:

DT (diphtheria toxin), DOX (doxycycline), h (hour), μl (microliter), μM (micromolar), mM (millimolar), mg (milligram), PPP (picropodophyllin).

### Materials and Methods

### Mice

All transgenic mice were previously described (Thorel et al, 2010, Nature 464(7292):1149-54; Yang et al, 2011, Genes Dev. 15; 25(16):1680-5; Kawamori et al, 2009, Cell Metab. 9(4):350-61).

### Diphtheria Toxin (DT), Doxycycline (DOX), S961, Picropodophyllin (PPP), Wortmannin and Insulin Treatments

DT (Sigma) was administrated by intra-peritoneal (i.p.) injections as previously described (Thorel et al., 2010, supra). DOX (1 mg/ml; Sigma) was added to the drinking water. S961 (Novo-Nordisk) (Vikram and Jena, 2010, Biochem Biophys Res Commun. 398(2):260-5) was injected intravenously (i.v.) twice a day at 200-nmol/kg-body weight, for 4 consecutive days. PPP (Santa-Cruz) and Wortmannin (Sigma) were injected i.p. once a day for 5 consecutive days at 10-mg/kg-body weight and 1-mg/kg-body weight, respectively. Mice received subcutaneous insulin pellets (Linbit) one week apart, if glycaemia exceeded 25 mM.

### Physiological Studies

Pancreatic glucagon and insulin dosages (immunoassays), gene expression analyses by real time PCR as well as histological and morphometric analyses were performed as described (Herrera et al, 1991, Development. 113(4):1257-65; Strom et al, 2007, Development. 134(15):2719-25).

### Immunofluorescence

Cryostat sections were 10 μm-thick. The antibodies used were: rabbit and guinea pig anti-Pdx1 (kind gift of C. Wright, 1/5000 and 1/750 respectively), guinea pig anti-porcine insulin (DAKO, 1/400), mouse anti-porcine glucagon (1/1000), mouse anti-somatostatin (BCBC Ab1985, 1/200), rabbit anti-GFP (Molecular Probes, 1/400), rabbit anti-Cpeptide1 (BCBC Ab1044, 1/500), rabbit anti-Cpeptide2 (BCBC Ab1042, 1/500). Secondary antibodies were coupled to either Alexa 405, 488, 647 (Molecular Probes), Cy3, Cy5 (Jackson Immunoresearch), or TRITC (Southern Biotech).

Sections were examined with a Leica TCS SPE confocal microscope.

### Isolation of Human Cell Fraction.

Human pancreatic islets were obtained from the Cell Isolation And Transplantation Center, University of Geneva. As described previously (Dorrell et al. 2008, Stem cell research, 1, 183-194) with few minor modifications, islets were incubated in accutase (Invitrogen) for 12 min. at 37° C. to prepare a single-cell suspension, followed by staining with α-cell surface antibodies (HPa1 or HPa2) and then with secondary antibodies. To obtain the pancreatic α-cell rich fraction, HPa1/2-positive cells were sorted on a FACSAria2 (BD Biosciences) or Moflo Astrios (Beckman Coulter) system. Single viable islet cells were gated by forward scatter, side scatter and pulse-width parameters as well as by negative staining for DAPI or PI. Establishment of the HPa1/2+ gate was based on the profile of sample stained without HPa1 or HPa2 antibody. Sorted cells were stained and analysed by using cytospin (Thermo Scientific) or Cunningham chambers as described in Bosco et al (Diabetes 2010, 59, 1202-1210).

### Example 1

### Massive α-Cell Conversion to Insulin Production is Driven by β-Cell Loss and Pdx1 Activity

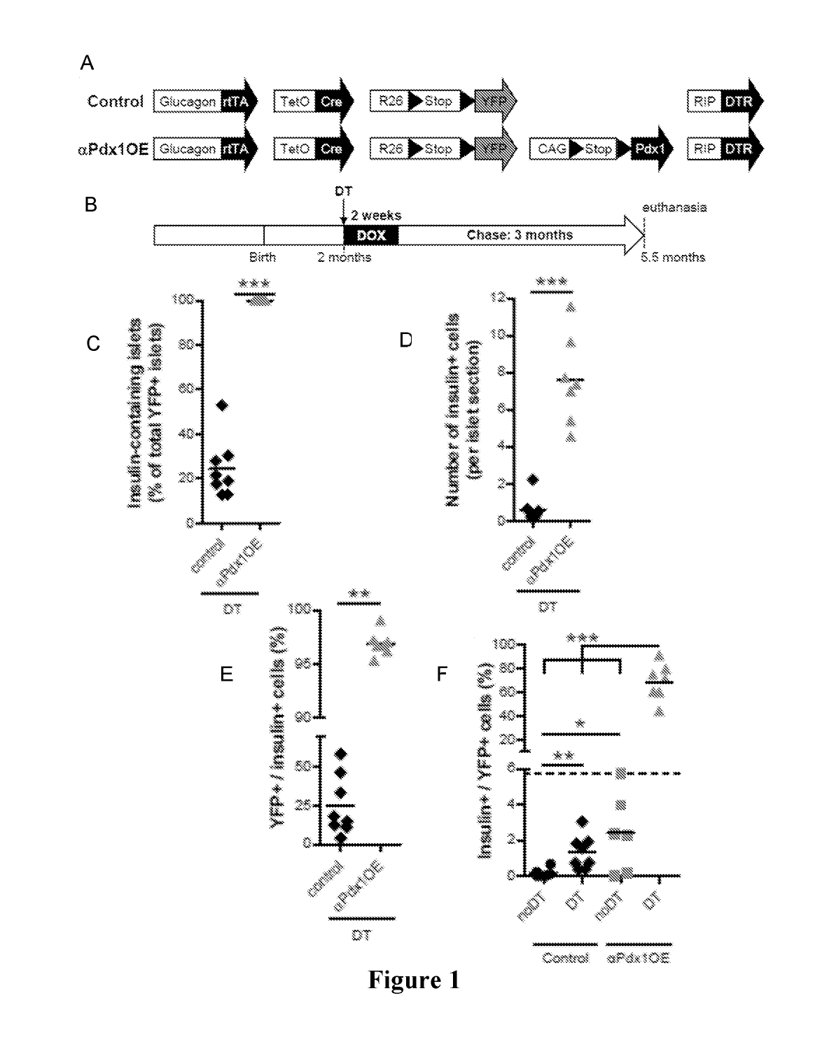

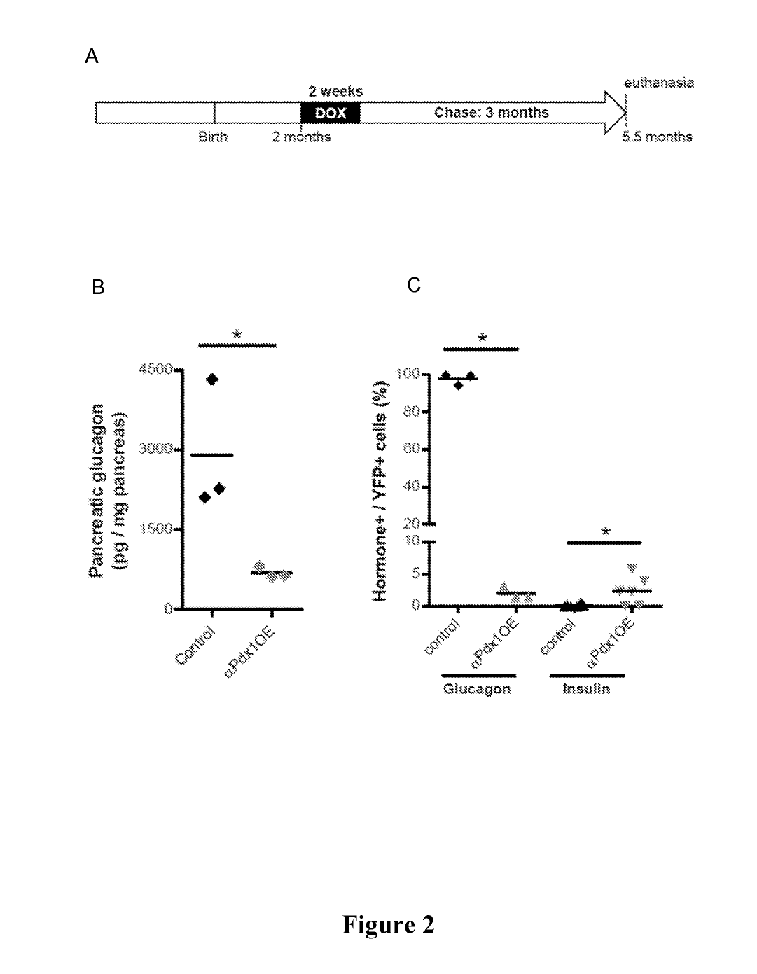

To determine whether promoting Pdx1 expression in all α-cells might facilitate their reprogramming, 5-fold transgenic mice, termed αPdx1OE, were generated allowing simultaneous, inducible and irreversible α-cell tracing and ectopic Pdx1 expression in α-cells upon doxycycline (DOX) administration (FIG. 1A). αPdx1OE mice bore the Glucagon-rtTA (α-cell specific reverse tetracycline transactivator expressor), TetO-cre (DOX-activated rtTA-dependent cre expressor), CAGSTOPfloxed-Pdx1 (cre-mediated Pdx1 expressor), rosa26-STOPfloxed-YFP (cremediated YFP reporter), RIP-DTR (DT-mediated β-cell killer) transgenes. Control mice lacked the CAG-STOPfloxed-Pdx1 transgene and thus allowed α-cell tracing only and not ectopic Pdx1 overexpression in α-cells (FIG. 1A). To assess whether Pdx1 promotes α-cell reprogramming to insulin production, 2 months-old mice were treated for 2 weeks with DOX (pulse) and euthanized 3 months after DOX ending (chase) (FIG. 2A). In healthy adult mice, sustained Pdx1 expression in α-cells results i) in inhibition of glucagon production (FIGS. 2B-C) and ii) marginal insulin production in a very small fraction (<3%) of adult α-cells (FIG. 2C). These results indicate that most adult α-cells are refractory to insulin production upon ectopic Pdx1 expression in condition of normal β-cell mass.

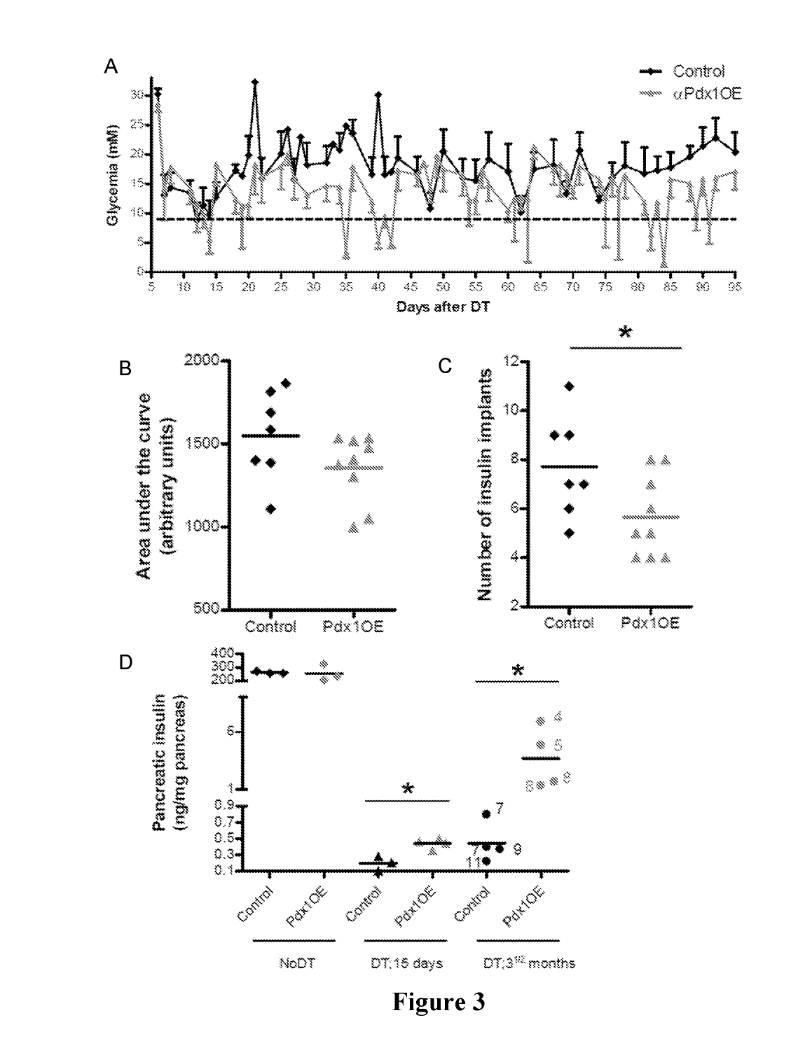

It was next tested whether reduced β-cell mass could represent a permissive condition for insulin production in α-cells expressing ectopically Pdx1. Near total β-cell removal was achieved by injecting mice with diphtheria toxin (DT, then after) as previously reported (Thorel et al., 2010, supra), and was followed by 2 weeks of DOX administration to induce irreversible α-cell labeling with YFP in both mice groups, and ectopic Pdx1 expression in αPdx1OE only (FIG. 1B). All mice became overtly hyperglycemic right after DT and were given insulin pellets once a week if glycemia exceed 25 mM to maintain diabetic mice alive. Interestingly, although αPdx1OE mice exhibited a trend toward lower glycemia as compared to control mice after DT (FIG. 3A-B), they required significantly less insulin pellets over the period of analysis (3.5 months post DT; FIG. 3C). In addition, while pancreatic insulin content remained unchanged in presence of intact β-cell mass, it recovered faster in αPdx1OE mice after DT-mediated β-cell loss (FIG. 3D). Remarkably, a direct negative correlation was observed between insulin content and insulin pellet requirement indicating that mice with higher pancreatic insulin content require less exogenous insulin to maintain their glycemia below 25 mM.

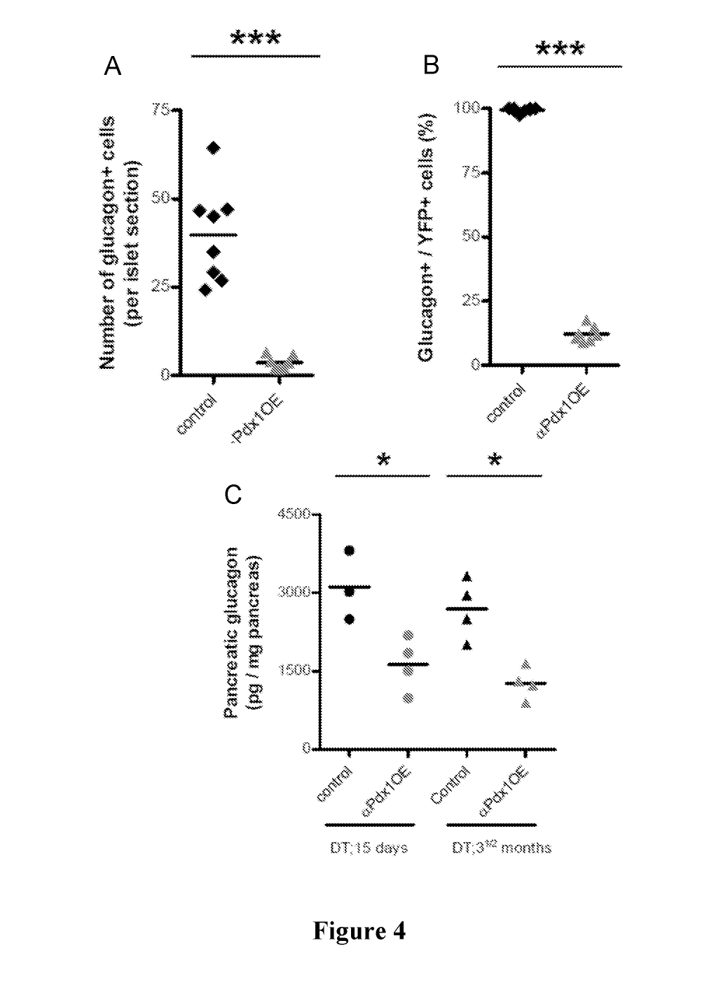

Altogether, these results suggest that the pancreas of αPdx1OE mice produce and secrete more insulin after massive β-cell loss as compared to those of controls. At the histological level, the vast majority of islets after DT are devoid of insulin-producing cells and are mostly composed of glucagon-expressing YFP-labeled α-cells in controls. By contrast, all α-cell containing islets contained insulin+ cells in αPdx1OE mice after DT (FIG. 1C), resulting in a significant increased insulin+ cell number as compared to controls (FIG. 1D). After β-cell loss, ectopic Pdx1 expression induced rapid insulin production (FIG. 3D) and glucagon inhibition (FIG. 4) in most YFP+α-cells. Both insulin genes, but not somatostatin, were induced in Pdx1-expressing adult α-cells after DT (not shown). After DT, only 25% of the very few insulin+ cells observed in control islets were YFP positive, i.e. reprogrammed α-cells. By contrast, nearly all insulin-producing cells (96%) derived from adult α-cells in αPdx1OE mice (FIG. 1E). While no adult α-cells were insulin producers in DT-untreated control mice, only a small fraction of adult α-cells (2-3%) reprogram to insulin production either i) after massive β-cell loss in control mice, or ii) upon ectopic Pdx1 expression in a situation of normal β-cell mass (FIG. 1F). By contrast, about 70% of adult α-cells having experienced cre-mediated recombination (YFP+) in αPdx1OE produced insulin upon synergistic ectopic Pdx1 expression and extreme β-cell loss (FIG. 1F).

Insulin production was also efficiently triggered if ectopic Pdx1 expression preceded DT-mediated β-cell loss (not shown). Importantly, insulin production in α-cells can be induced by ectopic Pdx1 expression in diabetic mice treated with exogenous insulin for at least several weeks. In agreement with this latter result and with the capacity of adult α-cells to reprogram to insulin production independently to circulating insulin and glucose levels in an islet autonomous dependent manner, ectopic Pdx1 expression induced efficient insulin production in α-cells from β-cell ablated islets transplanted under the kidney capsule of DT-insensitive (RIP-DTR negative) SCID mice (not shown). Taken together, these findings suggest that the vast majority of α-cells can reprogram to insulin production after near total β-cell loss (>99%) and ectopic Pdx1 expression, arguing against a heterogeneous α-cell population in terms of cell plasticity.

Parallel experiments showed that Nkx6.1 induction in mature α-cells does not block glucagon expression, contrary to Pdx1. However, after β-cell loss, Nkx6.1 activity resulted in simultaneous glucagon inhibition, insulin production and Pdx1 induction in Nkx6.1OE α-cells (not shown).

Together, these observations suggest that all α-cells have the plasticity to reprogram to insulin production if the conditions are appropriate, as revealed upon Pdx1 or Nkx6.1 activation in β-cell-depleted islets.

### Example 2

### Partial β-Cell Loss is Sufficient to Trigger Pdx1-Mediated Insulin Production in Adult α-Cells

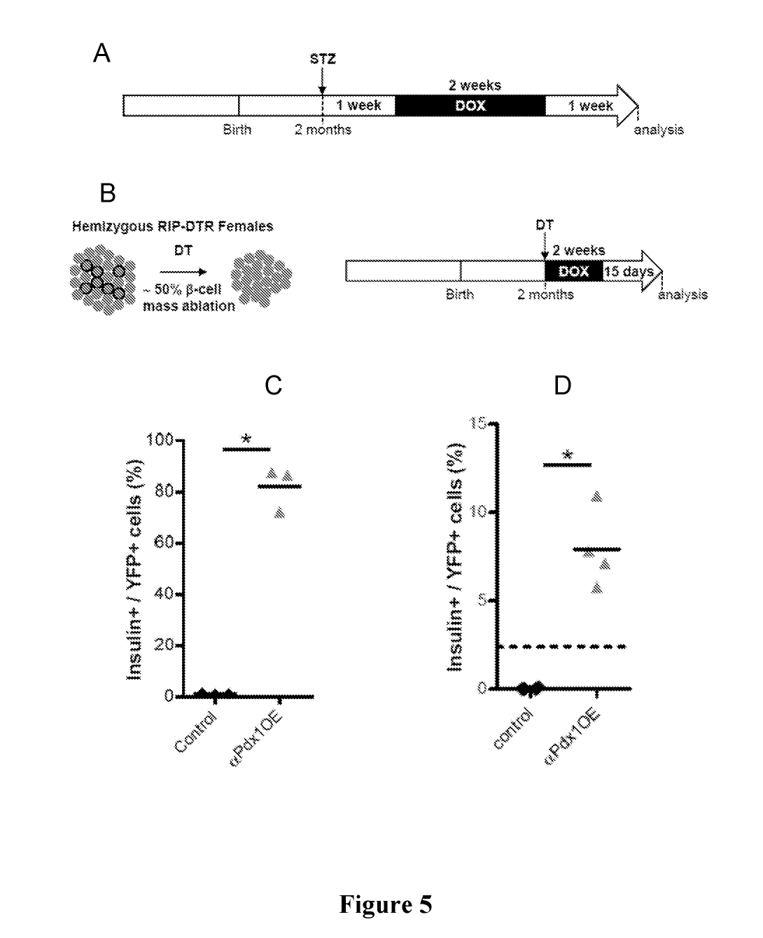

To determine whether Pdx1 can also trigger insulin production in adult α-cells after partial/suboptimal β-cell loss, control and αPdx1OE mice were injected with a single high dose (200 mg/kg body weight) of streptozotocin (STZ) to remove 80-90% of β-cells. After STZ, mice became hyperglycemic and were then treated with DOX for 2 weeks to trigger α-cell labeling, and Pdx1 expression in Pdx1OE mice (FIG. 5A). 1 month after STZ, a significant fraction of insulin-producing β-cells were still observed in islets of both mice groups, confirming that β-cell loss is not absolute upon STZ-mediated β-cell loss. Nearly all YFP-labeled α-cells were insulin negative in STZ-treated control mice. By contrast, most α-cells (>80%) expressing ectopically Pdx1 produce insulin after STZ in αPdx1OE islets (FIG. 5C). This suggests that STZ-mediated β-cell removal, despite not absolute, also prime/predispose α-cells to insulin production.

Next, it was tested whether ectopic Pdx1 expression triggers insulin production in α-cells when β-cell mass is reduced by only about 50%. In heterozygous RIP-DTR females, X-linked random inactivation restricts DTR expression to half of the β-cell population allowing 50% 3-cell ablation after DT administration (FIG. 5B). Heterozygous RIP-DTR control and αPdx1OE females were thus treated with DT and then with DOX for 2 weeks. Noteworthy, heterozygous RIP-DTR females remained normoglycemic after DT indicating that 50% β-cell mass is sufficient to ensure blood glucose homeostasis. 1 month after DT, YFP+α-cells expressing glucagon were insulin negative in control islets retaining 50% of their β-cell mass. Surprisingly, about 8% of YFP+α-cells were insulin producers in islets of normoglycemic αPdx1OE females after 50% β-cell ablation (FIG. 5D). However, no conversion of α-cells insulin production upon ectopic Pdx1 expression was observed during pregnancy, a condition of increased insulin demand (data not shown).

In conclusion, all these observations indicate that adult α-cell conversion to insulin production requires at least two events: DT- or STZ-mediated local β-cell loss, either extreme or partial, seems mandatory to predispose adult α-cells to insulin production. This “priming step” prepares all α-cells to insulin expression. All “primed” α-cells are then ready to produce insulin upon the ectopic transcriptional activity of Pdx1 (“triggering step”).

Importantly, the ability of pdx1 to trigger insulin expression in α-cells after STZ-mediated (3-cell loss strongly suggests that i) priming of α-cells after DT is not a bias of, or restricted to the RIP-DTR mouse model and, ii) insulin production can be efficiently induced irrespective to the mechanism of β-cell death, either by apoptosis after DT or by necrosis following STZ.

### Example 3

### Intra-Islet Insulin Deprivation Triggers α-Cell Priming to Insulin Production

Altogether these results suggest that β-cell loss seems mandatory to prime α-cells to insulin production. However, it is not clear whether α-cell priming is due to the physical β-cell loss or to the local deprivation of factor(s) secreted by β-cells, or both.

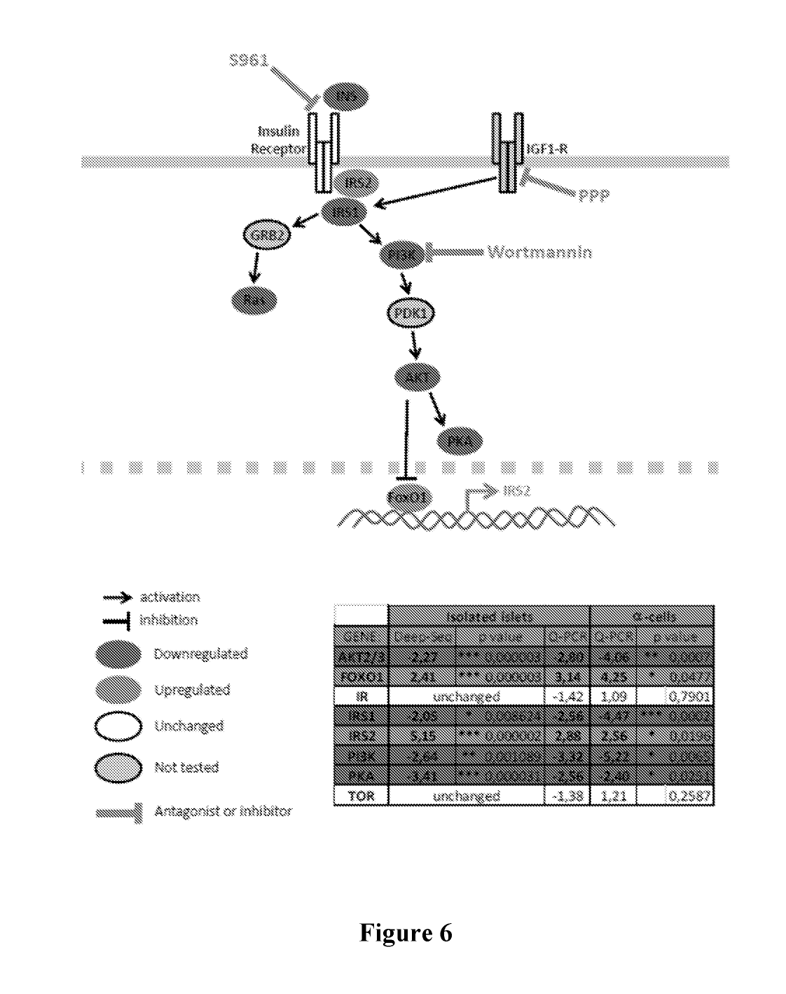

It was then tested whether local, intra-islet insulin deprivation (not systemic insulin level), which encompasses the massive loss of β-cells, might be the priming signal that triggers insulin production in α-cells expressing Pdx1. Systemic insulin is not the trigger of the observed α-cell plasticity, since α-cell conversion also occurs in healthy, normoglycemic mice. Gene expression analyses after near-total β-cell ablation revealed the rapid downregulation of key genes of the insulin-signaling cascade in β-cell-depleted islets. Indeed, mRNA levels of insulin and more downstream components of the insulin pathway, such as IRS 1, PI3K, Akt and PKA, were significantly reduced in islet extracts and in FACS-sorted α-cells 7 days after DT (FIG. 6). By contrast, FoxO1, which is negatively regulated by Akt was upregulated after DT in both isolated islets and α-cells. These results indicate that insulin signaling is blunted in α-cells after β-cell loss (FIG. 6, table).

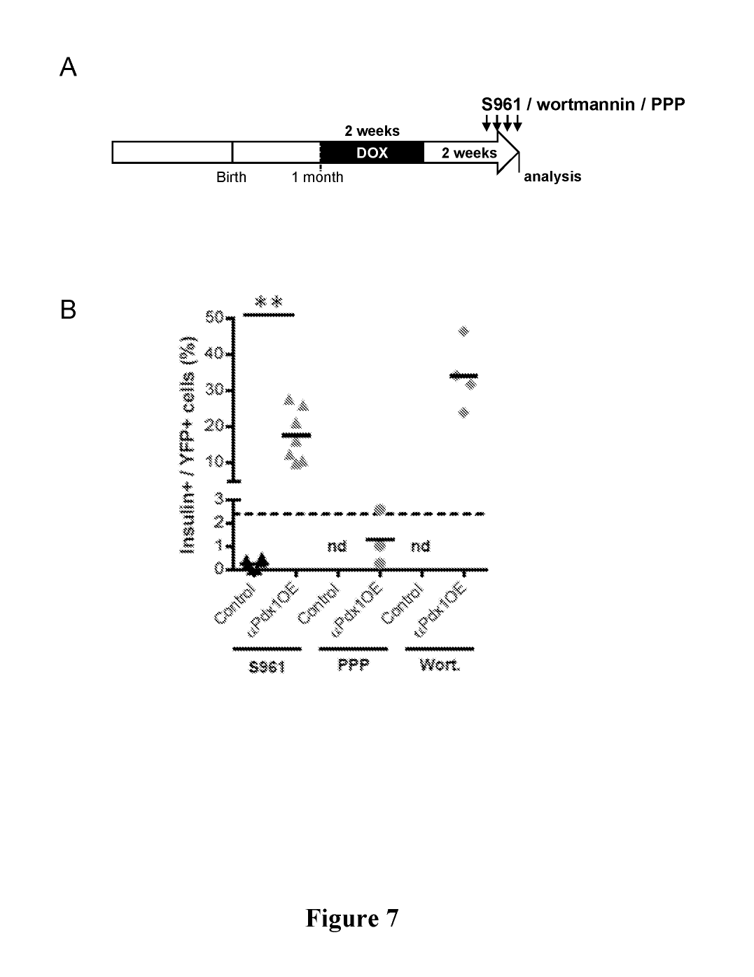

To test whether insulin deprivation in absence of β-cell loss can prime α-cells to insulin production, adult mice in which α-cells express Pdx1 (cPdx1OE) were treated with an insulin receptor antagonist, termed S961 (Novo Nordisk), to blunt insulin signaling in peripheral tissues as well as in pancreatic islets (FIG. 7A).

Peripheral action of S961 when administered in vivo induces hyperglycemia (not shown). A 5-day treatment to healthy adult mice having a normal β-cell mass led to insulin production in some 18% of Pdx1-expressing α-cells (FIG. 7B). In vivo administration of wortmannin, a PI3 Kinase inhibitor, but not picropodophyllin (PPP), a IGF-1R antagonist, also triggers insulin production with similar efficiency in Pdx1-expressing α-cells (FIG. 7B). Next, the insulin receptor was conditionally inactivated in adult α-cells to assess whether local inhibition of insulin signaling, exclusively at the level of α-cells, is sufficient to prime those cells to insulin production. Insulin receptor inactivation, YFP α-cell tracing and ectopic Pdx1 expression were induced in 1 month old mice upon DOX administration.

In conclusion, combined together, the above observations support a model in which the local constitutive release of insulin by the β-cells located in a given islet prevents priming α-cell to insulin production and thus α-cell conversion; therefore insulin acts as a paracrine repressor of α-cell plasticity.

### Example 4

### Lack of Insulin/IGF1 Signaling in α-Cells Predisposes Those Cells to Insulin Expression

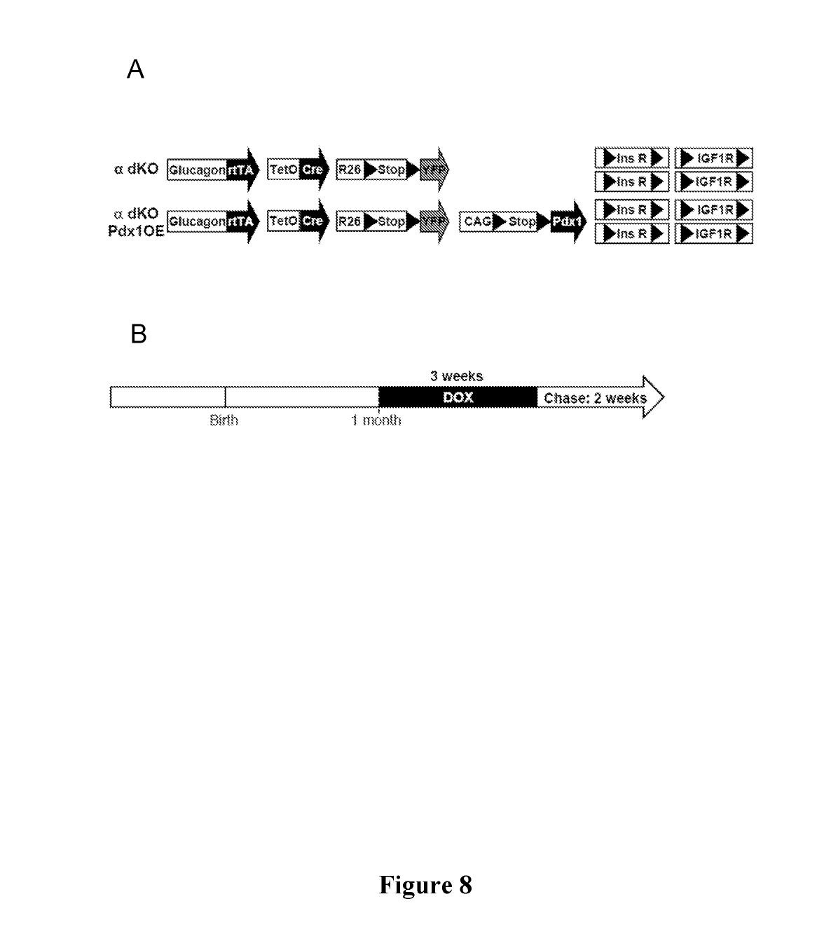

To determine whether α-cell-specific insulin/IGF1 deprivation, yet with a preserved β-cell mass, primes α-cells to producing insulin, transgenic mice termed “α-dKO” and “α-dKO-Pdx1OE” were generated to simultaneously inactivate insulin and IGF1 receptors (IR and IGF1R) in adult α-cells expressing or not Pdx1 (FIG. 8A). One-month-old mice were given DOX for 3 weeks to trigger α-cell-specific IR/IGF1R inactivation, Pdx1 expression and YFP-labeling (FIG. 8B).

Two weeks later, while α-cells in αdKO mice were glucagon⁺/insulin⁻, one-third of Pdx1-expressing α-cells in αdKO-Pdx1OE animals, which also have intact β-cell mass, were glucagon⁻/insulin⁺.

These results indicate that lack of insulin/IGF1 signaling in α-cells predisposes them to insulin expression.

### Example 5

### Human α-Cells can Reprogram to Insulin Production

It was further determined whether human α-cells also display the plasticity allowing insulin production.

Islets from human Type 1 Diabetic and Type 2 Diabetic patients were explored, and cells containing simultaneously secretory granules characteristic of 3- and α-cells were observed. The higher prevalence of bi-hormonal cells in diabetic patients supports the notion that β-cell loss and insulin resistance are conditions favoring insulin gene expression in α-cells.

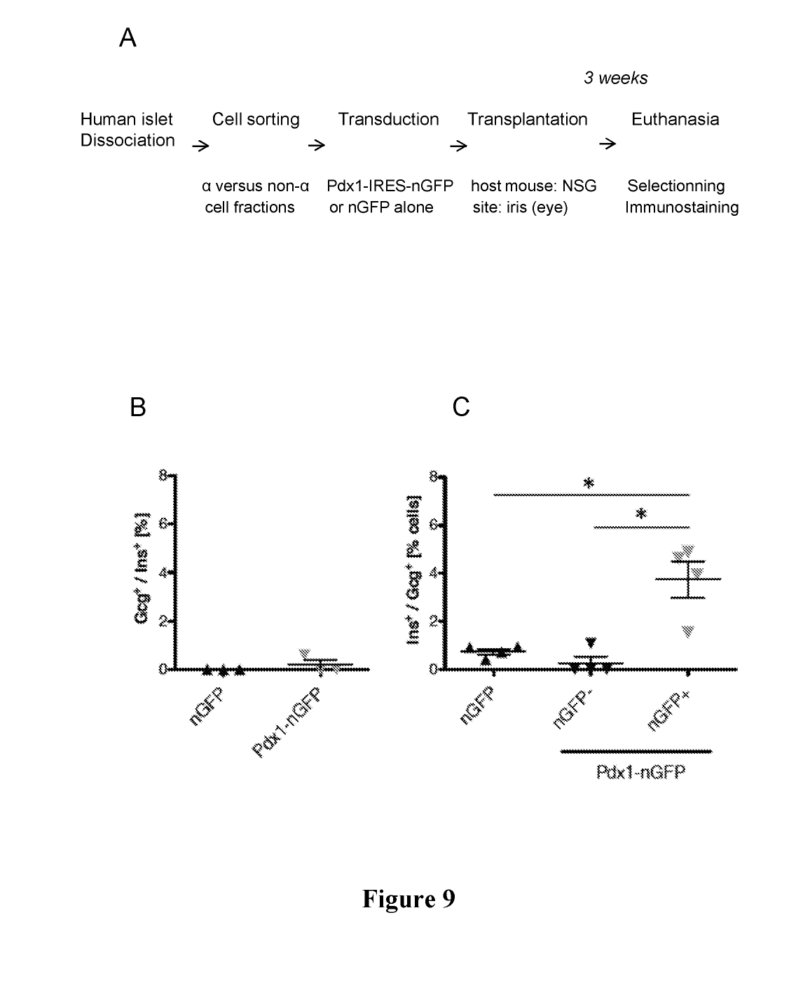

Thus, human α- and non-α-cell fractions were sorted by flow cytometry from non diabetic donors. The 2 groups of cells were separately transduced with either YFP- or YFP- and Pdx1-encoding adenoviral vectors as described in Zhou et al, 2008 (Nature 455: 627-632). Since islets and islet cells become unstable when maintained in vitro, transduced cells were transplanted on the iris of NSG mice as described in Shultz et al, 2007 (Nature reviews. Immunology 7, 118-130). Host mice were euthanized 3 weeks later, for analysis by immunofluorescence (FIG. 9A). No cell co-expressing insulin and glucagon were found in the non-α-cell fraction (FIG. 9B), or in lineage-traced α-cells in absence of Pdx1 (FIG. 9C). By contrast, when Pdx1 expression was induced in α-cells, the number of bihormonal-reprogrammed α-cells was significantly increased (FIG. 9C).

These results reveal that, like mouse α-cells, human α-cells from non-diabetic donors can undergo reprogramming to produce insulin in vivo, in normoglycemic mice, thus independently of glycemia and at extrapancreatic locations.

### Example 6

### Transplantation of Human α-Cells in Diabetic Mice

Human islets or purified islet cells are transferred to NSG-RIP-DTR mice made either diabetic (with DT or STZ) or insulin resistant (with S961 treatment, an insulin receptor antagonist), or to obese NSG-db/db mice. Human islets depleted from β-cells (after islet cell dissociation, FACS-sorting and re-aggregation/encapsulation without the β-cell fraction) are also transplanted, so as to mimic β-cell loss in human islets.

Human islet samples are transplanted under the kidney capsule or in the anterior chamber of the eye of NSG hosts (depending on the amount of islets or islet re-aggregated cells that are available).

In particular, to mimic β-cell loss in human islets, islet re-aggregates are reconstructed without β-cells, then encapsulated in alginate, and transferred into the abdominal cavity of NSG hosts. Before transplantation, human α-cells are transduced with adeno or lentiviral vectors expressing GFP (to lineage-trace the cells at analysis), and Pdx1, Nkx6.1 or other reprogramming factors so as to facilitate conversion.

Transplanted mice are further challenged with various compounds, such as TNFα (to impose an inflammatory stress), epigenetic modifiers (to facilitate cell plasticity through chromatin changes), or validated modulators of the signaling pathways promoting α- or δ-cell conversion.

The mice are monitored using metabolic parameters: Glycemia follow-up, glucose tolerance test (GTT) and human circulating C-peptide measurements are performed whenever appropriate to assess recovery of glycemia, and glucose-stimulated human insulin secretion. Mice are euthanized for analysis 1 or 3 months after transplantation; α- and δ-cell reprogramming, among other possible islet cell plasticity events, are determined by immunofluorescence using specific anti-insulin antibodies combined with anti-glucagon, anti-somatostatin and anti-GFP (human cell tracer) staining. Further characterization of the insulin-producing cells are performed using specific antibodies against maturity markers of functional β-cells (MafA, Nkx6.1, Ucn3, Glut2 . . . ).

Retrieval of alginate encapsulated islets/islet cells allows their RNA analyses (qPCR) and/or single cell gene profiling (fluidigm technology). Epigenetic studies (DNA methylation) are conducted when the amount of extracted genomic DNA is sufficient.

In summary, these experimental data show that α-cells become predisposed to insulin production if they cannot sense insulin properly. Although mainly described in peripheral tissues (adipose, liver, muscle), insulin resistance also occurs within islets in pathological situations associated with β- or α-cell dysfunction. Since insulin deficiency and resistance are characteristic of both Type 1 and Type 2 diabetes, α-cells in diabetic patients may thus be primed to insulin production. The bivalent active/repressive chromatin marks in human α-cells is compatible with these cells displaying high plasticity potential, which is further revealed in diabetic conditions. Such unforeseen cell conversion facilitation could be exploited in therapeutic strategies aimed at generating new insulin secreting cells by α-cell reprogramming. In particular, the counterintuitive transient induction of insulin antagonism in type 2 diabetic patients may help β-cell mass replenishment by promoting α-cell conversion. α-to-β-cell conversion, encompassing glucagon expression extinction, would also be beneficial for diabetics by limiting glucagon secretion, thus hepatic glucose mobilization, without defects due to α-cell deficit.

Sequence listing

mice Pdx-1 protein sequence
SEQ ID NO: 1
MNSEEQYYAA TQLYKDPCAF QRGPVPEFSA NPPACLYMGR QPPPPPPPQF TSSLGSLEQG
SPPDISPYEV PPLASDDPAG AHLHHHLPAQ LGLAHPPPGP FPNGTEPGGL EEPNRVQLPF
PWMKSTKAHA WKGQWAGGAY TAEPEENKRT RTAYTRAQLL ELEKEFLFNK YISRPRRVEL
AVMLNLTERH IKIWFQNRRM KWKKEEDKKR SSGTPSGGGG GEEPEQDCAV TSGEELLAVP
PLPPPGGAVP PGVPAAVREG LLPSGLSVSP QPSSIAPLRP QEPR
human Pdx-1 protein sequence
SEQ ID NO: 2
MNGEEQYYAA TQLYKDPCAF QRGPAPEFSA SPPACLYMGR QPPPPPPHPF PGALGALEQG
SPPDISPYEV PPLADDPAVA HLHHHLPAQL ALPHPPAGPF PEGAEPGVLE EPNRVQLPFP
WMKSTKAHAW KGQWAGGAYA AEPEENKRTR TAYTRAQLLE LEKEFLFNKY ISRPRRVELA
VMLNLTERHI KIWFQNRRMK WKKEEDKKRG GGTAVGGGGV AEPEQDCAVT SGEELLALPP
PPPPGGAVPP AAPVAAREGR LPPGLSASPQ PSSVAPRRPQ EPR
mouse Nkx 6.1 protein sequence
SEQ ID NO: 3
MLAVGAMEGP RQSAFLLSSP PLAALHSMAE MKTPLYPAAY PPLPTGPPSS SSSSSSSSSP
SPPLGSHNPG GLKPPAAGGL SSLGSPPQQL SAATPHGIND ILSRPSMPVA SGAALPSASP
SGSSSSSSSS ASATSASAAA AAAAAAAAAA ASSPAGLLAG LPRFSSLSPP PPPPGLYFSP
SAAAVAAVGR YPKPLAELPG RTPIFWPGVM QSPPWRDARL ACTPHQGSIL LDKDGKRKHT
RPTFSGQQIF ALEKTFEQTK YLAGPERARL AYSLGMTESQ VKVWFQNRRT KWRKKHAAEM
ATAKKKQDSE TERLKGTSEN EEDDDDYNKP LDPNSDDEKI TQLLKKHKSS GGSLLLHASE
AEGSS
human Nkx 6.1 protein sequence
SEQ ID NO: 4
MLAVGAMEGT RQSAFLLSSP PLAALHSMAE MKTPLYPAAY PPLPAGPPSS SSSSSSSSSP
SPPLGTHNPG GLKPPATGGL SSLGSPPQQL SAATPHGIND ILSRPSMPVA SGAALPSASP
SGSSSSSSSS ASASSASAAA AAAAAAAAAA SSPAGLLAGL PRFSSLSPPP PPPGLYFSPS
AAAVAAVGRY PKPLAELPGR TPIFWPGVMQ SPPWRDARLA CTPHQGSILL DKDGKRKHTR
PTFSGQQIFA LEKTFEQTKY LAGPERARLA YSLGMTESQV KVWFQNRRTK WRKKHAAEMA
TAKKKQDSET ERLKGASENE EEDDDYNKPL DPNSDDEKIT QLLKKHKSSS GGGGGLLLHA
SEPESSS
mouse Nkx 2.2 protein sequence
SEQ ID NO: 5
MSLTNTKTGF SVKDILDLPD TNDEDGSVAE GPEEESEGPE PAKRAGPLGQ GALDAVQSLP
LKSPFYDSSD NPYTRWLAST EGLQYSLHGL AASAPPQDSS SKSPEPSADE SPDNDKETQG
GGGDAGKKRK RRVLFSKAQT YELERRFRQQ RYLSAPEREH LASLIRLTPT QVKIWFQNHR
YKMKRARAEK GMEVTPLPSP RRVAVPVLVR DGKPCHALKA QDLAAATFQA GIPFSAYSAQ
SLQHMQYNAQ YSSASTPQYP TAHPLVQAQQ WTW
human Nkx 2.2 protein sequence
SEQ ID NO: 6
MSLTNTKTGF SVKDILDLPD TNDEEGSVAE GPEEENEGPE PAKRAGPLGQ GALDAVQSLP
LKNPFYDSSD NPYTRWLAST EGLQYSLHGL AAGAPPQDSS SKSPEPSADE SPDNDKETPG
GGGDAGKKRK RRVLFSKAQT YELERRFRQQ RYLSAPEREH LASLIRLTPT QVKIWFQNHR
YKMKRARAEK GMEVTPLPSP RRVAVPVLVR DGKPCHALKA QDLAAATFQA GIPFSAYSAQ
SLQHMQYNAQ YSSASTPQYP TAHPLVQAQQ WTW
mouse Pax 4 protein sequence
SEQ ID NO: 7
MQQDGLSSVN QLGGLFVNGR PLPLDTRQQI VQLAIRGMRP CDISRSLKVS NGCVSKILGR
YYRTGVLEPK CIGGSKPRLA TPAVVARIAQ LKDEYPALFA WEIQHQLCTE GLCTQDKAPS
VSSINRVLRA LQEDQSLHWT QLRSPAVLAP VLPSPHSNCG APRGPHPGTS HRNRTIFSPG
QAEALEKEFQ RGQYPDSVAR GKLAAATSLP EDTVRVWFSN RRAKWRRQEK LKWEAQLPGA
SQDLTVPKNS PGIISAQQSP GSVPSAALPV LEPLSPSFCQ LCCGTAPGRC SSDTSSQAYL
QPYWDCQSLL PVASSSYVEF AWPCLTTHPV HHLIGGPGQV PSTHCSNWP
human Pax 4 protein sequence
SEQ ID NO: 8
MHQDGISSMN QLGGLFVNGR PLPLDTRQQI VRLAVSGMRP CDISRILKVS NGCVSKILGR
YYRTGVLEPK GIGGSKPRLA TPPVVARIAQ LKGECPALFA WEIQRQLCAE GLCTQDKTPS
VSSINRVLRA LQEDQGLPCT RLRSPAVLAP AVLTPHSGSE TPRGTHPGTG HRNRTIFSPS
QAEALEKEFQ RGQYPDSVAR GKLATATSLP EDTVRVWFSN RRAKWRRQEK LKWEMQLPGA
SQGLTVPRVA PGIISAQQSP GSVPTAALPA LEPLGPSCYQ LCWATAPERC LSDTPPKACL
KPCWDCGSFL LPVIAPSCVD VAWPCLDASL AHHLIGGAGK ATPTHFSHWP
mouse Pax 6 protein sequence
SEQ ID NO: 9
MQNSHSGVNQ LGGVFVNGRP LPDSTRQKIV ELAHSGARPC DISRILQVSN GCVSKILGRY
YETGSIRPRA IGGSKPRVAT PEVVSKIAQY KRECPSIFAW EIRDRLLSEG VCTNDNIPSV
SSINRVLRNL ASEKQQMGAD GMYDKLRMLN GQTGSWGTRP GWYPGTSVPG QPTQDGCQQQ
EGGGENTNSI SSNGEDSDEA QMRLQLKRKL QRNRTSFTQE QIEALEKEFE RTHYPDVFAR
ERLAAKIDLP EARIQVWFSN RRAKWRREEK LRNQRRQASN TPSHIPISSS FSTSVYQPIP
QPTTPVSSFT SGSMLGRTDT ALTNTYSALP PMPSFTMANN LPMQPPVPSQ TSSYSCMLPT
SPSVNGRSYD TYTPPHMQTH MNSQPMGTSG TTSTGLISPG VSVPVQVPGS EPDMSQYWPR
LQ
human Pax 6 protein sequence
SEQ ID NO: 10
MQNSHSGVNQ LGGVFVNGRP LPDSTRQKIV ELAHSGARPC DISRILQVSN GCVSKILGRY
YETGSIRPRA IGGSKPRVAT PEVVSKIAQY KRECPSIFAW EIRDRLLSEG VCTNDNIPSV
SSINRVLRNL ASEKQQMGAD GMYDKLRMLN GQTGSWGTRP GWYPGTSVPG QPTQDGCQQQ
EGGGENTNSI SSNGEDSDEA QMRLQLKRKL QRNRTSFTQE QIEALEKEFE RTHYPDVFAR
ERLAAKIDLP EARIQVWFSN RRAKWRREEK LRNQRRQASN TPSHIPISSS FSTSVYQPIP
QPTTPVSSFT SGSMLGRTDT ALTNTYSALP PMPSFTMANN LPMQPPVPSQ TSSYSCMLPT
SPSVNGRSYD TYTPPHMQTH MNSQPMGTSG TTSTGLISPG VSVPVQVPGS EPDMSQYWPR
LQ
mouse MafA protein sequence
SEQ ID NO: 11
MAAELAMGAE LPSSPLAIEY VNDFDLMKFE VKKEPPEAER FCHRLPPGSL SSTPLSTPCS
SVPSSPSFCA PSPGTGGGAG GGGSAAQAGG APGPPSGGPG TVGGASGKAV LEDLYWMSGY
QHHLNPEALN LTPEDAVEAL IGSGHHGAHH GAHHPAAAAA YEAFRGQSFA GGGGADDMGA
GHHHGAHHTA HHHHSAHHHH HHHHHHGGSG HHGGGAGHGG GGAGHHVRLE ERFSDDQLVS
MSVRELNRQL RGFSKEEVIR LKQKRRTLKN RGYAQSCRFK RVQQRHILES EKCQLQSQVE
QLKLEVGRLA KERDLYKEKY EKLAGRGGPG GAGGAGFPRE PSPAQAGPGA AKGAPDFFL
human MafA protein sequence
SEQ ID NO: 12
MAAELAMGAE LPSSPLAIEY VNDFDLMKFE VKKEPPEAER FCHRLPPGSL SSTPLSTPCS
SVPSSPSFCA PSPGTGGGGG AGGGGGSSQA GGAPGPPSGG PGAVGGTSGK PALEDLYWMS
GYQHHLNPEA LNLTPEDAVE ALIGSGHHGA HHGAHHPAAA AAYEAFRGPG FAGGGGADDM
GAGHHHGAHH AAHHHHAAHH HHHHHHHHGG AGHGGGAGHH VRLEERFSDD QLVSMSVREL
NRQLRGFSKE EVIRLKQKRR TLKNRGYAQS CRFKRVQQRH ILESEKCQLQ SQVEQLKLEV
GRLAKERDLY KEKYEKLAGR GGPGSAGGAG FPREPSPPQA GPGGAKGTAD FFL
mouse Ngn3 protein sequence
SEQ ID NO: 13
MAPHPLDALT IQVSPETQQP FPGASDHEVL SSNSTPPSPT LIPRDCSEAE VGDCRGTSRK
LRARRGGRNR PKSELALSKQ RRSRRKKAND RERNRMHNLN SALDALRGVL PTFPDDAKLT
KIETLRFAHN YIWALTQTLR IADHSFYGPE PPVPCGELGS PGGGSNGDWG SIYSPVSQAG
NLSPTASLEE FPGLQVPSSP SYLLPGALVF SDFL
human Ngn3 protein sequence
SEQ ID NO: 14
MTPQPSGAPT VQVTRETERS FPRASEDEVT CPTSAPPSPT RTRGNCAEAE EGGCRGAPRK
LRARRGGRSR PKSELALSKQ RRSRRKKAND RERNRMHNLN SALDALRGVL PTFPDDAKLT
KIETLRFAHN YIWALTQTLR IADHSLYALE PPAPHCGELG SPGGSPGDWG SLYSPVSQAG
SLSPAASLEE RPGLLGATFS ACLSPGSLAF SDFL
mouse NeuroD1 protein sequence
SEQ ID NO: 15
MTKSYSESGL MGEPQPQGPP SWTDECLSSQ DEEHEADKKE DELEAMNAEE DSLRNGGEEE
EEDEDLEEEE EEEEEEEDQK PKRRGPKKKK MTKARLERFK LRRMKANARE RNRMHGLNAA
LDNLRKVVPC YSKTQKLSKI ETLRLAKNYI WALSEILRSG KSPDLVSFVQ TLCKGLSQPT
TNLVAGCLQL NPRTFLPEQN PDMPPHLPTA SASFPVHPYS YQSPGLPSPP YGTMDSSHVF
HVKPPPHAYS AALEPFFESP LTDCTSPSFD GPLSPPLSIN GNFSFKHEPS AEFEKNYAFT
MHYPAATLAG PQSHGSIFSS GAAAPRCEIP IDNIMSFDSH SHHERVMSAQ LNAIFHD
human NeuroD1 protein sequence
SEQ ID NO: 16
MTKSYSESGL MGEPQPQGPP SWTDECLSSQ DEEHEADKKE DDLETMNAEE DSLRNGGEEE
DEDEDLEEEE EEEEEDDDQK PKRRGPKKKK MTKARLERFK LRRMKANARE RNRMHGLNAA
LDNLRKVVPC YSKTQKLSKI ETLRLAKNYI WALSEILRSG KSPDLVSFVQ TLCKGLSQPT
TNLVAGCLQL NPRTFLPEQN QDMPPHLPTA SASFPVHPYS YQSPGLPSPP YGTMDSSHVF
HVKPPPHAYS AALEPFFESP LTDCTSPSFD GPLSPPLSIN GNFSFKHEPS AEFEKNYAFT
MHYPAATLAG AQSHGSIFSG TAAPRCEIPI DNIMSFDSHS HHERVMSAQL NAIFHD
Insulin receptor antagonist S661 amino acid sequence
SEQ ID NO: 17
GSLDESFYDW FERQLGGGSG GSSLEEEWAQ IQCEVWGRGC PSX
wherein X is Tyr with a C-terminal carboxylic acid group replaced
by an amide group, and wherein the two Cys at positions 33 and 40
are joined by a disulfide bond.
Insulin receptor antagonist S961 amino acid sequence
SEQ ID NO: 18
GSLDESFYDW FERQLGGGSG GSSLEEEWAQ IQCEVWGRGC PSY
wherein the two Cys at positions 33 and 40 are joined by a
disulfide bond.

1-8. (canceled)

9. A method of inducing insulin production in non-β-cells comprising the step of stimulating the insulin production of non-β-cells expressing at least one transcription factor characteristic of pancreatic β-cells by blocking the insulin signaling pathway.

10. (canceled)

11. The method according to claim 9, wherein said non-β-cells are selected from the group consisting of pancreatic α-cells, δ-cells, PP cells, ε-cells, neuroendocrine cells associated with the digestive tract, and peripheral cells.

12. The method according to claim 9, wherein blocking the insulin signaling pathway is carried out ex vivo by contacting said non-β-cells with an antagonist of the insulin signaling pathway.

13. The method according to claim 9, wherein blocking the insulin signaling pathway is carried out in vivo by administering an antagonist of the insulin signaling pathway to a diabetic subject.

14. The method according to claim 9, wherein said antagonist of the insulin signaling pathway is selected from the group consisting of: S961, S661, a covalent insulin dimer crosslinked between the two B29 lysines (B29-B′29), Wortmannin, PX-866, SF1126, GDC-0941, XL-147, XL-765, D-87503, D-106669, GSK-615, CAL-101, NVP-BEZ235, LY294002, Buparlisib (also called BKM-120), GDC-0032, BAY 80-6946, IPI-145, BYL-719, BGT-226, PF-04691502, GDC-0980, GSK-2126458, and PF-05212384.

15. The method according to claim 9, comprising the steps of:
a) modifying non-β-cells by inducing the expression of at least one transcription factor characteristic of pancreatic β-cells; and
b) stimulating the insulin production of the modified non-β-cells obtained in step a) by blocking the insulin signaling pathway.

16. The method according to claim 15, wherein said transcription factor is selected from the group consisting of Pdx-1, Nkx 6.1, Nkx 2.2, Pax 4, Pax 6, MafA, Ngn3, and NeuroD1.

17. A method of screening a compound for its ability to inhibit the insulin signaling pathway comprising:
a) exposing non-β-cells expressing at least one transcription factor characteristic of β-cells to a test compound;
b) determining the number of said cells which are insulin producing cells in presence and in absence of the test compound; and
c) comparing the two values of number of insulin producing cells determined in step b),
wherein a number of insulin producing cells that is higher in presence of the test compound compared to the number determined in absence of the test compound is indicative of a test compound able to inhibit the insulin signaling pathway.

18. A method of predicting the susceptibility of a diabetic subject to a treatment of diabetes comprising the administration of a therapeutically effective amount of an antagonist of the insulin signaling pathway in a subject in need thereof, comprising a step of detecting the expression of at least one transcription factor characteristic of pancreatic β-cells in non-β-cells from said subject.

19. The method according to claim 18, wherein said transcription factor is selected from the group consisting of Pdx-1, Nkx 6.1, Nkx 2.2, Pax 4, Pax 6, MafA, Ngn3, and NeuroD1.

20. A method of preventing and/or treating diabetes comprising the administration of a therapeutically effective amount of an antagonist of the insulin signaling pathway in a subject in need thereof.

21. The method according to claim 20, wherein said antagonist is selected from the group consisting of: S961, S661, a covalent insulin dimer crosslinked between the two B29 lysines (B29-B′29), Wortmannin, PX-866, SF1126, GDC-0941, XL-147, XL-765, D-87503, D-106669, GSK-615, CAL-101, NVP-BEZ235, LY294002, Buparlisib (also called BKM-120), GDC-0032, BAY 80-6946, IPI-145, BYL-719, BGT-226, PF-04691502, GDC-0980, GSK-2126458, and PF-05212384.

22. The method according to claim 20, wherein said antagonist is S961 of SEQ ID NO: 18.

23. The method according to claim 20 comprising administering a composition comprising (i) an antagonist of the insulin signaling pathway and (ii) non-β-cells modified by transfection of a nucleic acid encoding at least one transcription factor characteristic of pancreatic β-cells in a subject in need thereof.

24. The method according to claim 23, wherein said non-β-cells are selected from the group consisting of pancreatic α-cells, δ-cells, PP cells, ε-cells, neuroendocrine cells associated with the digestive tract, and peripheral cells.

25. The method according to claim 23, wherein said transcription factor is selected from the group consisting of Pdx-1, Nkx 6.1, Nkx 2.2, Pax 4, Pax 6, MafA, Ngn3, and NeuroD1.

26. The method according to claim 20 wherein the subject in need thereof is a diabetic subject identified by a method of predicting the susceptibility of a diabetic subject to a treatment of diabetes comprising the administration of a therapeutically effective amount of an antagonist of the insulin signaling pathway in a subject in need thereof, comprising a step of detecting the expression of at least one transcription factor characteristic of pancreatic β-cells in non-β-cells from said subject.

27. The method according to claim 20 wherein the subject in need thereof is a diabetic subject identified by a method of predicting the susceptibility of a diabetic subject to a treatment of diabetes comprising the administration of a therapeutically effective amount of an antagonist of the insulin signaling pathway in a subject in need thereof, comprising a step of detecting the expression of at least one transcription factor characteristic of pancreatic β-cells in non-β-cells from said subject.

28. A pharmaceutical composition comprising an antagonist of the insulin signaling pathway and non-β-cells modified by transfection of a nucleic acid encoding at least one transcription factor characteristic of pancreatic β-cells.

29. The pharmaceutical formulation according to claim 28 wherein said antagonist is selected from the group consisting of: S961, S661, a covalent insulin dimer crosslinked between the two B29 lysines (B29-B′29), Wortmannin, PX-866, SF1126, GDC-0941, XL-147, XL-765, D-87503, D-106669, GSK-615, CAL-101, NVP-BEZ235, LY294002, Buparlisib (also called BKM-120), GDC-0032, BAY 80-6946, IPI-145, BYL-719, BGT-226, PF-04691502, GDC-0980, GSK-2126458, and PF-05212384.


SEQUENCE LISTING (18 sequences)

SEQ ID NO: 1 - protein, 284 aa, Mus musculus
MNSEEQYYAATQLYKDPCAFQRGPVPEFSANPPACLYMGRQPPPPPPPQFTSSLGSLEQGSPPDISPYEVPPLASDDPAGAHLHHHLPAQLGLAHPPPGPFPNGTEPGGLEEPNRVQLPFPWMKSTKAHAWKGQWAGGAYTAEPEENKRTRTAYTRAQLLELEKEFLFNKYISRPRRVELAVMLNLTERHIKIWFQNRRMKWKKEEDKKRSSGTPSGGGGGEEPEQDCAVTSGEELLAVPPLPPPGGAVPPGVPAAVREGLLPSGLSVSPQPSSIAPLRPQEPR

SEQ ID NO: 2 - protein, 283 aa, Homo sapiens
MNGEEQYYAATQLYKDPCAFQRGPAPEFSASPPACLYMGRQPPPPPPHPFPGALGALEQGSPPDISPYEVPPLADDPAVAHLHHHLPAQLALPHPPAGPFPEGAEPGVLEEPNRVQLPFPWMKSTKAHAWKGQWAGGAYAAEPEENKRTRTAYTRAQLLELEKEFLFNKYISRPRRVELAVMLNLTERHIKIWFQNRRMKWKKEEDKKRGGGTAVGGGGVAEPEQDCAVTSGEELLALPPPPPPGGAVPPAAPVAAREGRLPPGLSASPQPSSVAPRRPQEPR

SEQ ID NO: 3 - protein, 365 aa, Mus musculus
MLAVGAMEGPRQSAFLLSSPPLAALHSMAEMKTPLYPAAYPPLPTGPPSSSSSSSSSSSPSPPLGSHNPGGLKPPAAGGLSSLGSPPQQLSAATPHGINDILSRPSMPVASGAALPSASPSGSSSSSSSSASATSASAAAAAAAAAAAAAASSPAGLLAGLPRFSSLSPPPPPPGLYFSPSAAAVAAVGRYPKPLAELPGRTPIFWPGVMQSPPWRDARLACTPHQGSILLDKDGKRKHTRPTFSGQQIFALEKTFEQTKYLAGPERARLAYSLGMTESQVKVWFQNRRTKWRKKHAAEMATAKKKQDSETERLKGTSENEEDDDDYNKPLDPNSDDEKITQLLKKHKSSGGSLLLHASEAEGSS

SEQ ID NO: 4 - protein, 367 aa, Homo sapiens
MLAVGAMEGTRQSAFLLSSPPLAALHSMAEMKTPLYPAAYPPLPAGPPSSSSSSSSSSSPSPPLGTHNPGGLKPPATGGLSSLGSPPQQLSAATPHGINDILSRPSMPVASGAALPSASPSGSSSSSSSSASASSASAAAAAAAAAAAAASSPAGLLAGLPRFSSLSPPPPPPGLYFSPSAAAVAAVGRYPKPLAELPGRTPIFWPGVMQSPPWRDARLACTPHQGSILLDKDGKRKHTRPTFSGQQIFALEKTFEQTKYLAGPERARLAYSLGMTESQVKVWFQNRRTKWRKKHAAEMATAKKKQDSETERLKGASENEEEDDDYNKPLDPNSDDEKITQLLKKHKSSSGGGGGLLLHASEPESSS

SEQ ID NO: 5 - protein, 273 aa, Mus musculus
MSLTNTKTGFSVKDILDLPDTNDEDGSVAEGPEEESEGPEPAKRAGPLGQGALDAVQSLPLKSPFYDSSDNPYTRWLASTEGLQYSLHGLAASAPPQDSSSKSPEPSADESPDNDKETQGGGGDAGKKRKRRVLFSKAQTYELERRFRQQRYLSAPEREHLASLIRLTPTQVKIWFQNHRYKMKRARAEKGMEVTPLPSPRRVAVPVLVRDGKPCHALKAQDLAAATFQAGIPFSAYSAQSLQHMQYNAQYSSASTPQYPTAHPLVQAQQWTW

SEQ ID NO: 6 - protein, 273 aa, Homo sapiens
MSLTNTKTGFSVKDILDLPDTNDEEGSVAEGPEEENEGPEPAKRAGPLGQGALDAVQSLPLKNPFYDSSDNPYTRWLASTEGLQYSLHGLAAGAPPQDSSSKSPEPSADESPDNDKETPGGGGDAGKKRKRRVLFSKAQTYELERRFRQQRYLSAPEREHLASLIRLTPTQVKIWFQNHRYKMKRARAEKGMEVTPLPSPRRVAVPVLVRDGKPCHALKAQDLAAATFQAGIPFSAYSAQSLQHMQYNAQYSSASTPQYPTAHPLVQAQQWTW

SEQ ID NO: 7 - protein, 349 aa, Mus musculus
MQQDGLSSVNQLGGLFVNGRPLPLDTRQQIVQLAIRGMRPCDISRSLKVSNGCVSKILGRYYRTGVLEPKCIGGSKPRLATPAVVARIAQLKDEYPALFAWEIQHQLCTEGLCTQDKAPSVSSINRVLRALQEDQSLHWTQLRSPAVLAPVLPSPHSNCGAPRGPHPGTSHRNRTIFSPGQAEALEKEFQRGQYPDSVARGKLAAATSLPEDTVRVWFSNRRAKWRRQEKLKWEAQLPGASQDLTVPKNSPGIISAQQSPGSVPSAALPVLEPLSPSFCQLCCGTAPGRCSSDTSSQAYLQPYWDCQSLLPVASSSYVEFAWPCLTTHPVHHLIGGPGQVPSTHCSNWP

SEQ ID NO: 8 - protein, 350 aa, Homo sapiens
MHQDGISSMNQLGGLFVNGRPLPLDTRQQIVRLAVSGMRPCDISRILKVSNGCVSKILGRYYRTGVLEPKGIGGSKPRLATPPVVARIAQLKGECPALFAWEIQRQLCAEGLCTQDKTPSVSSINRVLRALQEDQGLPCTRLRSPAVLAPAVLTPHSGSETPRGTHPGTGHRNRTIFSPSQAEALEKEFQRGQYPDSVARGKLATATSLPEDTVRVWFSNRRAKWRRQEKLKWEMQLPGASQGLTVPRVAPGIISAQQSPGSVPTAALPALEPLGPSCYQLCWATAPERCLSDTPPKACLKPCWDCGSFLLPVIAPSCVDVAWPCLDASLAHHLIGGAGKATPTHFSHWP

SEQ ID NO: 9 - protein, 422 aa, Mus musculus [sequence omitted]

SEQ ID NO: 10 - protein, 422 aa, Homo sapiens [sequence omitted]

SEQ ID NO: 11 - protein, 359 aa, Mus musculus
MAAELAMGAELPSSPLAIEYVNDFDLMKFEVKKEPPEAERFCHRLPPGSLSSTPLSTPCSSVPSSPSFCAPSPGTGGGAGGGGSAAQAGGAPGPPSGGPGTVGGASGKAVLEDLYWMSGYQHHLNPEALNLTPEDAVEALIGSGHHGAHHGAHHPAAAAAYEAFRGQSFAGGGGADDMGAGHHHGAHHTAHHHHSAHHHHHHHHHHGGSGHHGGGAGHGGGGAGHHVRLEERFSDDQLVSMSVRELNRQLRGFSKEEVIRLKQKRRTLKNRGYAQSCRFKRVQQRHILESEKCQLQSQVEQLKLEVGRLAKERDLYKEKYEKLAGRGGPGGAGGAGFPREPSPAQAGPGAAKGAPDFFL

SEQ ID NO: 12 - protein, 353 aa, Homo sapiens
MAAELAMGAELPSSPLAIEYVNDFDLMKFEVKKEPPEAERFCHRLPPGSLSSTPLSTPCSSVPSSPSFCAPSPGTGGGGGAGGGGGSSQAGGAPGPPSGGPGAVGGTSGKPALEDLYWMSGYQHHLNPEALNLTPEDAVEALIGSGHHGAHHGAHHPAAAAAYEAFRGPGFAGGGGADDMGAGHHHGAHHAAHHHHAAHHHHHHHHHHGGAGHGGGAGHHVRLEERFSDDQLVSMSVRELNRQLRGFSKEEVIRLKQKRRTLKNRGYAQSCRFKRVQQRHILESEKCQLQSQVEQLKLEVGRLAKERDLYKEKYEKLAGRGGPGSAGGAGFPREPSPPQAGPGGAKGTADFFL

SEQ ID NO: 13 - protein, 214 aa, Mus musculus
MAPHPLDALTIQVSPETQQPFPGASDHEVLSSNSTPPSPTLIPRDCSEAEVGDCRGTSRKLRARRGGRNRPKSELALSKQRRSRRKKANDRERNRMHNLNSALDALRGVLPTFPDDAKLTKIETLRFAHNYIWALTQTLRIADHSFYGPEPPVPCGELGSPGGGSNGDWGSIYSPVSQAGNLSPTASLEEFPGLQVPSSPSYLLPGALVFSDFL

SEQ ID NO: 14 - protein, 214 aa, Homo sapiens
MTPQPSGAPTVQVTRETERSFPRASEDEVTCPTSAPPSPTRTRGNCAEAEEGGCRGAPRKLRARRGGRSRPKSELALSKQRRSRRKKANDRERNRMHNLNSALDALRGVLPTFPDDAKLTKIETLRFAHNYIWALTQTLRIADHSLYALEPPAPHCGELGSPGGSPGDWGSLYSPVSQAGSLSPAASLEERPGLLGATFSACLSPGSLAFSDFL

SEQ ID NO: 15 - protein, 357 aa, Mus musculus
MTKSYSESGLMGEPQPQGPPSWTDECLSSQDEEHEADKKEDELEAMNAEEDSLRNGGEEEEEDEDLEEEEEEEEEEEDQKPKRRGPKKKKMTKARLERFKLRRMKANARERNRMHGLNAALDNLRKVVPCYSKTQKLSKIETLRLAKNYIWALSEILRSGKSPDLVSFVQTLCKGLSQPTTNLVAGCLQLNPRTFLPEQNPDMPPHLPTASASFPVHPYSYQSPGLPSPPYGTMDSSHVFHVKPPPHAYSAALEPFFESPLTDCTSPSFDGPLSPPLSINGNFSFKHEPSAEFEKNYAFTMHYPAATLAGPQSHGSIFSSGAAAPRCEIPIDNIMSFDSHSHHERVMSAQLNAIFHD

SEQ ID NO: 16 - protein, 356 aa, Homo sapiens
MTKSYSESGLMGEPQPQGPPSWTDECLSSQDEEHEADKKEDDLETMNAEEDSLRNGGEEEDEDEDLEEEEEEEEEDDDQKPKRRGPKKKKMTKARLERFKLRRMKANARERNRMHGLNAALDNLRKVVPCYSKTQKLSKIETLRLAKNYIWALSEILRSGKSPDLVSFVQTLCKGLSQPTTNLVAGCLQLNPRTFLPEQNQDMPPHLPTASASFPVHPYSYQSPGLPSPPYGTMDSSHVFHVKPPPHAYSAALEPFFESPLTDCTSPSFDGPLSPPLSINGNFSFKHEPSAEFEKNYAFTMHYPAATLAGAQSHGSIFSGTAAPRCEIPIDNIMSFDSHSHHERVMSAQLNAIFHD

SEQ ID NO: 17 - protein, 43 aa, Artificial Sequence
  Insulin receptor antagonist S661
GSLDESFYDWFERQLGGGSGGSSLEEEWAQIQCEVWGRGCPSX

SEQ ID NO: 18 - protein, 43 aa, Artificial Sequence
  Insulin receptor antagonist S961
GSLDESFYDWFERQLGGGSGGSSLEEEWAQIQCEVWGRGCPSY